(19) United States
(12) Patent Application Publication
(10) Pub. No.: US 2005/0181033 A1
(43) Pub. Date: Aug. 18, 2005

(54) Method for delivering interferons to the intradermal compartment

(76) Inventors: John P. Dekker III, Cary, NC (US); John A. Mikszta, Durham, NC (US); Ronald J. Pettis, Cary, NC (US); Paul G. Alchas, Franklin Lakes, NJ (US)

Correspondence Address: JONES DAY, 222 EAST 41ST ST, NEW YORK, NY, 10017, US

(21) Appl. No.: 11/075,276

(22) Filed: Mar. 8, 2005

Related U.S. Application Data
(63) Continuation-in-part of application No. 10/803,746, filed on Mar. 17, 2004.
Application No. 10/803,746 is a continuation-in-part of application No. 09/893,746, filed on Jun. 29, 2001.
Application No. 09/893,746 is a continuation-in-part of application No. 09/606,909, filed on Jun. 29, 2000.
(60) Provisional application No. 60/551,293, filed on Mar. 8, 2004.

(51) Int. Cl. A61K 38/21; A61K 9/70

(52) U.S. Cl. 424/449; 424/85.5; 424/85.6; 424/85.7

(57) ABSTRACT

The present invention relates to methods and devices for intradermal delivery of substances, preferably therapeutic substances by targeting the substance to the intradermal compartment of a subject's skin. Substances delivered in accordance with the methods of the invention have an improved clinical utility and therapeutic efficacy relative to other drug delivery methods including intramuscular, and subcutaneous delivery. The present invention provides benefits and improvements over conventional drug delivery methods including but not limited to, improved pharmacokinetics and bioavailability.

This application is a continuation-in-part of U.S. application Ser. No. 10/803,746, filed on Mar. 17, 2004, which is is a continuation-in-part of U.S. application Ser. No. 09/893,746 filed Jun. 29, 2001, which is a continuation-in-part of U.S. application Ser. No. 09/606,909 filed Jun. 29, 2000, each of which is incorporated herein by reference in its entirety. Additionally this application claims the benefit of priority of U.S. Provisional Application Ser. No. 60/551,293 filed on Mar. 8, 2004 under 35 U.S.C. § 119(e) which is also incorporated herein by reference in its entirety.

### 1. FIELD OF THE INVENTION

The present invention relates to methods and devices for intradermal delivery of substances, preferably therapeutic substances by depositing the substance into the intradermal compartment of a subject's skin. Substances delivered in accordance with the methods of the invention have an improved clinical utility and therapeutic efficacy relative to other drug delivery methods including intramuscular, and subcutaneous delivery. The present invention provides benefits and improvements over conventional drug delivery methods including but not limited to, improved pharmacokinetics and improved bioavailability.

### 2. BACKGROUND OF THE INVENTION

### 2.1 Drug Delivery

The importance of efficiently and safely administering pharmaceutical substances such as diagnostic agents and drugs has long been recognized. Although an important consideration for all pharmaceutical substances, obtaining adequate bioavailability of large molecules such as proteins that have arisen out of the biotechnology industry has recently highlighted this need to obtain efficient and reproducible absorption (Cleland et al., Curr. Opin. Biotechnol. 12: 212-219, 2001). The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e., delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular, the papillary dermis has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue which has been far more easily and predictably targeted than the dermis has been. On the other hand, large molecules such as proteins are typically not well absorbed through the capillary epithelium regardless of the degree of vascularity so that one would not have expected to achieve a significant absorption advantage over subcutaneous administration by the more difficult to achieve intradermal administration, even for large molecules.

One approach to administration beneath the surface to the skin and into the region of the intradermal space has been routinely used in the Mantoux tuberculin test. ‘In this procedure, a purified protein derivative is injected at a shallow angle to the skin surface using a 27 or 30 gauge needle (Flynn et al., Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of substances.

Some groups have reported on systemic administration by what has been characterized as “intradermal” injection. In one such report, a comparison study of subcutaneous and what was described as “intradermal” injection was performed (Autret et al., Therapie 46:5-8, 1991). The pharmaceutical substance tested was calcitonin, a protein of a molecular weight of about 3600. Although it was stated that the drug was injected intradermally, the injections used a 4 mm needle pushed up to the base at an angle of 60. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al., J. Pharm. Sci. 82:1175-1178, 1993). This would have resulted in injection to a dept of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus, there remains a continuing need for efficient and safe methods and devices for administration of pharmaceutical substances.

### SUMMARY OF THE INVENTION

The present disclosure relates to a new parenteral administration method based on directly targeting the dermal space whereby such method dramatically alters the pharmacokinetic (PK) and pharmacodynamic (PD) parameters of administered substances. By the use of “direct intradermal (ID) administration” means hereafter referred to as “dermal-access means”, for example, using microneedle-based injection and infusion systems (or other means to accurately target the intradermal space), the pharmacokinetics of many substances including drugs and diagnostic substances, which are especially protein and peptide hormones, can be altered when compared to traditional parental administration routes of subcutaneous and intravenous delivery. These findings are pertinent not only to microdevice-based injection means, but other delivery methods such as needless or needle-free ballistic injection of fluids or powders into the ID space, Mantoux-type ID injection, enhanced iontophoresis through microdevices, and direct deposition of fluid, solids, or other dosing forms into the skin. Disclosed is a method to increase the rate of uptake for parenterally-administered drugs without necessitating IV access. One significant beneficial effect of this delivery method is providing a shorter Tₘₐₓ (time to achieve maximum blood concentration of the drug). Potential corollary benefits include higher maximum concentrations for a given unit dose (Cₘₐₓ), higher bioavailability, more rapid uptake rates, more rapid onset of pharmacodynamics or biological effects, and reduced drug depot effects. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to subcutaneous, intramuscular or other non-IV parenteral means of drug delivery.

The present invention provides intradermal administration of substances, preferably therapeutic substances by depositing the substance into the intradermal compartment of a subject's skin. Substances delivered in accordance with the methods of the invention have an improved clinical utility and therapeutic efficacy relative to other drug delivery methods including intramuscular, and subcutaneous delivery. The present invention provides benefits and improvements over conventional drug delivery methods including but not limited to, improved pharmacokinetics, reduction of undesired immune responses, and reduced immunogenicity. Substances delivered in accordance with the methods of the invention result in an immune response no greater than the immune response observed when delivered by other routes including intramuscular, and subcutaneous.

The methods of the invention result in a similar type or similar level of immune response as conventional methods of drug delivery including intramuscular and subcutaneous delivery. In accordance with the invention, an immune response is considered to be of the same type if the immune responses being compared are both humoral, cellular, tolerogenic, Th1 or Th2 mediated immune responses. In accordance with the invention, the level of an immune response may be qualitatively or quantitatively determined using standard methods in the art, such as ELISA, FACS, and immunoprecipitation techniques. The invention encompasses a method for administration of a substance to a subject's skin comprising delivering the substance into an intradermal compartment of the subject's skin, wherein the substance results in an immune response no greater than when the substance is delivered intramuscularly.

In a specific embodiment, substances delivered to the intradermal compartment in accordance with the methods of the invention result in an antibody response, against the substance similar to that obtained when the substance is delivered intramuscularly or subcutaneously, i.e., similar mean antibody titer as measured using for example an ELISA assay. In a more preferred embodiment, substances delivered to the intradermal compartment in accordance with the methods of the invention result in an antibody response against the substance which is less than the antibody response when the substance is delivered intramuscularly or subcutaneously, i.e., lower mean antibody titer as measured using for example an ELISA assay. In same embodiments the methods of the invention result in a same type of immune response as that when the substance is delivered to the subcutaneous or intramuscular compartment.

The methods of the invention result in minimal to moderate, preferably minimal irritation at the injection site based on histopathological evaluations such as those disclosed herein. In most preferred embodiments, the irritation at the injection site is not altered upon chronic administration. The methods of the invention result in no acute or chronic irritation at the site of injection as observed visually.

Substances delivered to the intradermal compartment in accordance with the methods of the invention have improved bioavailability and improved therapeutic efficacy relative to other drug delivery methods including intramuscular and subcutaneous delivery. The invention is based, in part, on the unexpected discovery by the inventors that when therapeutic proteins are targeted to the intradermal compartment, their immunogenicity remains unchanged or is reduced as compared to conventional modes of administration of such therapeutic substances including IM and SC administration. This discovery is particularly unexpected given that the intradermal compartment comprises immune cells, including dendritic cells and antigen presenting cells, which typically allow direct access of an antigenic or immunogenic agent for eliciting a desired immune response. Delivery of therapeutic proteins using the conventional modes of delivery can result in unwanted immune responses upon administration, especially upon chronic administration. Thus, the methods of the invention are particularly effective for administration of therapeutic substances to which the induction of an immune response would not be beneficial to the therapeutic effect of the substance to be delivered.

As used herein, intradermal is intended to mean administration of a substance into the dermis in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e., the papillary dermis or in the upper portion of the relatively less vascular reticular dermis such that the substance readily diffuses into the papillary dermis. It is believed that placement of a substance predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of macromolecular and/or hydrophobic substances. Although not intending to be bound by a particular mechanism of action, placement of the substance predominately at greater depths and/or into the lower portion of the reticular dermis results in the substance being slowly absorbed in the less vascular reticular dermis or in the subcutaneous region either of which would result in reduced absorption of macromolecular and/or hydrophobic substances. The controlled delivery of a substance in this dermal space below the papillary dermis in the reticular dermis, but sufficiently above the interface between the dermis and the subcutaneous tissue, should enable an efficient outward migration of the substance to the undisturbed vascular and lymphatic microcapillary bed, ie., in the papillary dermis, where it can be absorbed into systemic circulation via these microcapillaries without being sequestered in transit by any other cutaneous tissue compartment.

Substances that can be delivered to the intradermal compartment of a subject's skin in accordance with the present invention include pharmaceutically or biologically active substances include diagnostic agents, drugs, and other substances which provide therapeutic or health benefits such as for example nutraceuticals. The invention encompasses the administration of any protein, particularly a therapeutic protein, and all salts, polymorphs, analogs, derivatives, fragments, mimetics, and peptides thereof. Substances that are particularly suited for the methods of the invention are those which can benefit from a reduced risk of unwanted immune response as encountered by conventional modes of administration including IM and SC. Substances for use in the methods of the invention are preferably formulated so that they do not induce an immune response when delivered to the intradermal compartment, or if an immune response is induced, it is of a type that is not associated with neutralization of the drug or other undesired outcomes. Preferably formulations of the invention contain no additives, including but not limited to excipients and adjuvants, which enhance the antigenicity or immunogenicity of the substances when delivered to the intradermal compartment. Preferably, the formulations of the invention do not reside as a depot or extended release formulation in the skin. Although not intending to be bound by any mechanism of action substances delivered in accordance with the methods of the invention based, in part, on their formulations, do not enhance presentation and/or availability of the substance to the immune cells of the intradermal compartment, rather they are rapidly systemically distributed thus bypassing the immune system.

The methods of the invention are particularly useful for chronic administration of substances, e.g., therapeutic proteins which typically result in an unwanted immune response upon chronic administration by other routes. Preferred substances for use in the methods of the invention are, cytokines (e.g., interferons, including interferon alpha and beta) chemokines, hormones (e.g., insulin, human growth hormone), immunomodulatory and therapeutic proteins including but not limited to monoclonal antibodies and fusion proteins comprised of antibodies, antigen-binding domains of antibodies or other fragments thereof.

The present invention provides for targeting and deposition of a substance into the intradermal compartment of the skin. Directly targeting the dermal space in accordance with the methods of the invention results in improved pharmacokinetic (PK) and pharmacodynamic (PD) parameters of administered substances.

The present invention encompasses any device for accurately and selectively targeting the intradermal compartment of a subject's skin. The nature of the device used is not critical as long as it penetrates the skin of the subject to the targeted depth within the intradermal space without passing through it. Preferably the device penetrates the skin to a depth of 0.5 to 2 mm, preferably to a depth of 1-2 mm.

In some embodiments, the present invention encompasses delivering a substance into the intradermal compartment of a subject's skin using a device that comprises at least one needle, preferably a microneedle. Preferably, the needle has a length sufficient to penetrate the intradermal compartment and an outlet at a depth within the intradermal compartment so that the substance is delivered and deposited therein.

The invention encompasses pharmaceutical formulations comprising one or more substances for intradermal delivery. In some embodiments, the formulations containing a substance of the invention comprises a therapeutically or prophylactically effective amount of the substance. In other embodiments, the formulations of the invention comprise one or more other additives. The formulations to be administered according to the methods of the present invention may be in any form suitable for intradermal delivery. Substances delivered according to the invention may be PEGylated, or otherwise altered chemically so as to improve the pharmacokinetics of uptake of the substance and/or to reduce the immunogenicity of the substance relative to its unaltered form.

The invention encompasses intradermal delivery of substances as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

### 3.1 Definitions

As used herein, “intradermal” is intended to mean administration of a substance into the dermis in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e., the papillary dermis, or in the upper portion of the relatively less vascular reticular dermis such that the substance readily diffuses into the papillary dermis.

As used herein, “intradermal delivery” means the delivery of agents to the intradermal compartment as described by Pettis et al. in WO 02/02179 A1 (PCT/US01/20782) and U.S. application Ser. No. 09/606,909; each of which is incorporated herein by reference in their entireties

As used herein, “ID Mantoux delivery” refers to the traditional ID Mantoux tuberculin test where an agent is injected at a shallow angle to the skin surface using a 27 or 30 gauge needle and standard syringe (see, e.g., Flynn et al, Chest 106: 1463-5, 1994, which is incorporated herein by reference in its entirety). The Mantoux technique involves inserting the needle into the skin laterally, then “snaking” the needle further into the ID tissue. The technique is known to be quite difficult to perform and requires specialized training. A degree of imprecision in placement of the delivery results in a significant number of false negative test results. Moreover, the method involves a localized injection to elicit a response at the site of injection and the Mantoux approach has not led to the use of intradermal injection for systemic administration of agents. When delivering the agent by ID Mantoux, the needle is substantially parallel to the surface at the skin, preferably at an angle of no more that 30° and best described as being between 10° and 15°. Mantoux deposition of injectate, when performed correctly, results in an elliptical pattern with the injectate in the SC and ID tissues. ID deposition as described herein results in a rounded deposition pattern of the injectate contained in the ID tissue. When delivering an agent by the ID Mantoux method, the insertion angle of the needle is preferably at a 15° angle parallel to the skin surface. Histological examination of the injection site after an agent has been administered by ID Mantoux results in an elliptical wheal deposition pattern, and a substantial part of the agent delivered gets deposited into the SC compartment of the skin rather than the ID compartment. ID Mantoux method is typically used clinically in diagnostic procedures such as sentinal node biopsy procedures for detection of tumors, however the method is quite difficult to perform and requires specialized training and has numerous limitations including, sites of administration, complications of injection, and patient discomfort.

As used herein “subcutaneous delivery” refers to deposition of an agent into the subcutaneous layer of a subject's skin at a depth greater than 2.5 mm.

By “improved pharmacokinetics” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}).

As used herein, the term “immunogenicity” refers to the property of a substance (e.g., foreign objects, microorganisms, drugs, antigens, proteins, peptides, polypeptides, nucleic acids, DNA, RNA, etc.) being able to evoke an immune response within an organism. Immunogenicity depends partly upon the size of the substance in question, partly upon how unlike the host molecules is the substance and partly upon the form in which the substance is presented to the immune system. Highly conserved proteins tend to have rather low immunogenicity.

As used herein, the term “unwanted immune response” means the natural immune response of the subject receiving a substance of this invention, where the substance is not intended to provoke such response when administered. Examples of unwanted immune responses that may be prevented using the methods of the invention include, but are not limited to, IgE-mediated hypersensitivity, with the risk of local and/or systemic anaphylactic reaction as described, for instance, after parenteral injection of insulin or heparin and many other drugs based on having a protein or a polysaccharide as the active ingredient; antibody-mediated cytotoxic hypersensitivity as well as immune complex mediated hypersensitivity that cause systemic adverse events, such as kidney and/or liver and/or microvascular alteration due to the deposition of circulating immune complexes; cell-mediated hypersensitivity with the risk of inducing delayed type reaction at the injection site and immune neutralization of the active ingredient, or any systemic adverse event, such as thrombocytopenia induced by heparin treatment; the formation of antibodies that neutralize the effect of the drug, regardless of whether or not such neutralization is accompanied with clinical symptoms.

As used herein, the terms “disorder” and “disease” are used interchangeably to refer to a condition in a subject. “Disease” or “disorder” carry their ordinary meaning in the art, in that they refers to an interruption or disorder of body functions, systems or organs. It encompasses any disturbance of function, structure or both, resulting from a genetic or embryologica failure in development or from exogenous factors such as poison, trauma, or disease. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

### 4. DESCRIPTION OF THE DRAWINGS

FIG. 1 shows a time course of plasma insulin levels of intradermal versus subcutaneous bolus administration of fast-acting.

FIG. 2 shows a time course of blood glucose levels of intradermal versus subcutaneous bolus administration of fast-acting insulin.

FIG. 3 shows a comparison of bolus ID dosing of fast-acting versus regular insulin.

FIG. 4 shows the effects of different intradermal injection depths for bolus dosing of fast-acting insulin on the time course of insulin levels

FIG. 5 shows a comparison of the time course of insulin levels for bolus dosing of long-acting insulin administered subcutaneously or intradermally.

FIGS. 6 and 7 show a comparison of the pharmacokinetic availability and the pharmacodynamic results of granulocyte colony stimulating factor delivered intradermally with a single needle or three point needle array, subcutaneously, or intravenously.

FIGS. 8, 9 and 10 show a comparison of low molecular weight heparin intradermal delivery by bolus, short duration, long duration infusion with comparison to subcutaneous infusion.

FIG. 11 shows the antibody titer of the 2^{nd} an 4^{th} week bleed after chronic ID and IM administration of rat-derived recombinant IFN-β to female Wistar rats.

FIG. 12 shows the antibody titer of the 6^{th} and 12^{th} week bleed after chronic ID and IM administration of rat-derived recombinant IFN-β to female Wistar rats.

FIG. 13 shows the antibody titer of the 24^{th} week bleed after chronic ID and IM administration of rat-derived recombinant IFN-β.

FIG. 14 shows the IgG1 antibody isotype level at weeks 2 and 24 after chronic ID and IM administration of rat-derived recombinant IFN-β.

FIG. 15 shows the IgG2b antibody isotype level at week 24 after chronic ID and IM administration of rat-derived recombinant IFN-β.

FIGS. 16A-B show injection site photos of IFN-β to female Wistar rats. These photos were taken immediately post-injection.

A. This panel shows an intramuscular injection. The back of a rat's leg is shown. The injection was made into the hamstring muscle. The needle insertion point is circled.

B. This panel is an intradermal injection on the rat's back. The “bleb” is circled.

FIG. 17 NEEDLE DEVICE. An exploded perspective illustration of a needle assembly designed according to this invention.

FIG. 18 NEEDLE DEVICE. A partial cross-sectional illustration of the embodiment of FIG. 17.

FIG. 19 NEEDLE DEVICE. Embodiment of FIG. 18 attached to a syringe.

FIG. 20 shows the results of neutralizing IFN-β specific antibody titer studies at weeks 2 and 24 post-immunization.

FIG. 21 HISTOPATHOLOGICAL ANALYSIS AT WEEK 2 POST INJECTION—COMPARISON OF INTRADERMAL AND INTRAMUSCULAR INJECTIONS

FIG. 22 ID INJECTION TECHNIQUE. A perspective view of one technique for making an ID injection.

FIG. 23 ID INJECTION TECHNIQUE. A perspective view of a 2^{nd} technique for making an ID injection.

FIG. 24 ID INJECTION TECHNIQUE. A perspective view of a 3rd technique for making an ID injection.

FIG. 25 ID INJECTION TECHNIQUE. A perspective view of a 4^{th} technique for making an ID injection.

### 5. DETAILED DESCRIPTION OF THE INVENTION

The invention provides intradermal administration of substances, preferably therapeutic substances by targeting the substance to the intradermal compartment of a subject's skin. Substances delivered in accordance with the methods of the invention have an improved pharmacokinetics, improved bioavailability and therapeutic efficacy relative to conventional drug delivery methods including intramuscular, and subcutaneous delivery. The present invention provides benefits and improvements over conventional drug delivery methods including but not limited to, improved pharmacokinetics, reduction of undesired immune responses and reduced immunogenicity, or induction of a similar type or level of immune response as via conventional drug delivery methods while, concomitantly, providing improved pharmacokinetics of drug uptake. The present invention is based, in part, on the unexpected discovery by the inventors that when therapeutic proteins are targeted to the intradermal compartment, their immunogenicity is unaltered or reduced compared to conventional modes of administration of such therapeutic substances, including IM and SC administration. This discovery is particularly unexpected given that the intradermal compartment comprises immune cells, including dendritic cells and antigen presenting cells, which typically allow direct access of an antigenic or immunogenic agent for eliciting a desired immune response. Therapeutic proteins result in unwanted immune responses upon administration using the conventional modes of delivery, especially upon chronic administration. However, the inventors have demonstrated for the first time that targeting therapeutic proteins to the intradermal compartment, results in improved bioavailability and unaltered or reduced immunogenicity compared to the conventional routes of administration. Specifically, the inventors have found that chronic administration of therapeutic proteins to the intradermal compartment results in an immune response which is no greater than when the substances are delivered intramuscularly or subcutaneously.

Substances that can be delivered to the intradermal compartment of a subject's skin in accordance with the present invention include pharmaceutically or biologically active substances including include diagnostic agents, drugs, and other substances which provide therapeutic or health benefits such as for example nutraceuticals. The invention encompasses the administration of any protein, particularly a therapeutic protein, and all salts, polymorphs, analogs, derivatives, fragments, mimetics, and peptides thereof. Substances that are particularly suited for the methods of the invention are those which can benefit from a reduced risk of unwanted immune response as encountered by conventional modes of administration including IM and SC. Substances delivered in accordance with the methods of the invention result in an immune response no greater than the immune response observed when delivered by other routes including intramuscular, and subcutaneous.

The methods of the invention result in a similar type or similar level of immune response as conventional methods of drug delivery including intramuscular and subcutaneous delivery. In accordance with the invention, an immune response is considered to be of the same type if the immune responses being compared are both humoral, cellular, tolerogenic, Th1 or Th2 mediated immune responses. In accordance with the invention, the level of an immune response may be qualitatively or quantitatively determined using standard methods in the art, such as ELISA, FACS, and immunoprecipitation techniques. The invention encompasses a method for administration of a substance to a subject's skin comprising delivering the substance into an intradermal compartment of the subject's skin, wherein the substance results in an immune response no greater than when the substance is delivered intramuscularly.

In a specific embodiment, substances delivered to the intradermal compartment in accordance with the methods of the invention result in an antibody response, against the substance similar to that obtained when the substance is delivered intramuscularly or subcutaneously, i.e., similar mean antibody titer as measured using for example an ELISA assay. In a more preferred embodiment, substances delivered to the intradermal compartment in accordance with the methods of the invention result in an antibody response against the substance which is less than the antibody response when the substance is delivered intramuscularly or subcutaneously, i.e., lower mean antibody titer as measured using for example an ELISA assay. In same embodiments the methods of the invention result in a same type of immune response as that when the substance is delivered to the subcutaneous or intramuscular compartment.

The methods of the invention result in minimal to moderate, preferably minimal irritation at the injection site based on histopathological evaluations such as those disclosed herein. In most preferred embodiments, the irritation at the injection site is not altered upon chronic administration. The methods of the invention result in no acute or chronic irritation at the site of injection as observed visually.

The methods of the invention are particularly useful for chronic administration of substances, e.g., therapeutic proteins which often result in an unwanted immune response upon chronic administration by other routes. Preferred substances for use in the methods of the invention are, cytokines (e.g., interferons, including interferon alpha and beta) chemokines, immunomodulatory and therapeutic proteins including but not limited to monoclonal antibodies and fusion proteins comprised of antibodies, antigen-binding domains of antibodies or other fragments thereof (e.g., human growth hormone, insulin). Substances for use in the methods of the invention are preferably formulated so that they do not induce an immune response when delivered to the intradermal compartment. Preferably, formulations of the invention contain no additives, including but not limited to excipients and adjuvants, which enhance the antigenicity or immunogenicity of the substances when delivered to the intradermal compartment. Although not intending to be bound by any mechanism of action substances delivered in accordance with the methods of the invention based, in part, on their formulations, do not enhance presentation and/or availability of the substance to the immune cells of the intradermal compartment, rather they are rapidly systemically distributed thus bypassing the immune system. Preferably, formulations of the invention contain no additives, including but not limited to excipients and adjuvants, which enhance the antigenicity or immunogenicity of the substances when delivered to the intradermal compartment. Although not intending to be bound by any mechanism of action substances delivered in accordance with the methods of the invention based, in part, on their formulations, do not enhance presentation and/or availability of the substance to the immune cells of the intradermal compartment, rather they are rapidly systemically distributed thus bypassing the immune system. The method of the invention preferably result in a similar type or level of immune response as via conventional drug delivery methods concomitalt, providing improved pharmacokinetics of drug uptake.

Substances administered using the methods of the present invention yield pharmacokinetics superior to and more clinically desirable than that obtained for the same substance administered by conventional methods of delivery including SC and IM. Administering a substance in accordance with the methods of the invention increases the rate of uptake for parenterally-administered drugs without necessitating IV access. One significant beneficial effect of this delivery method is providing a shorter Tₘₐₓ, i.e., time to achieve maximum blood concentration of the drug. Potential corollary benefits include higher maximum concentrations for a given unit dose (Cₘₐₓ), higher bioavailability, more rapid uptake rates, more rapid onset of pharmacodynamics or biological effects, and reduced drug depot effects. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to subcutaneous, intramuscular or other non-IV parenteral means of drug delivery. By bioavailability is meant the total amount of a given dosage that reached the blood compartment. This is generally measured as the area under the curve in a plot of concentration vs. time. By “lag time” is meant the delay between the administration of a compound and time to measure or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood or target tissue concentration necessary to realize a biological effect. Tₘₐₓ and Cₘₐₓ can be determined by visual inspection of graphical results and can often provide sufficient information to compare two methods administration of a compound. However, numerical values can be determined more precisely by analysis using kinetic models as described below and/or other means known to those of skill in the art.

Thus, in accordance with the invention “improved pharmacokinetics” means that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}). By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration-such-as, for ex-ample,—subcutaneous administration or intramuscular administration. In such comparisons, it is preferable, although not necessarily essential, that administration into the intradermal layer and administration into the reference site such as subcutaneous administration involve the same dose levels, i.e., the same amount and concentration of drug as well as the same carrier vehicle and the same rate of administration in terms of amount and volume per unit time. Thus, for example, administration of a given pharmaceutical substance into the dermis at a concentration such as 100 μg/mL and rate of 100 μL per minute over a period of 5 minutes would, preferably, be compared to administration of the same pharmaceutical substance into the subcutaneous space at the same concentration of 100 μg/mL and rate of 100 μL per minute over a period of 5 minutes.

The enhanced absorption profile is particularly evident for substances which are not well absorbed when injected subcutaneously such as, for example, macromolecules and/or hydrophobic substances. Macromolecules are, in general, not well absorbed subcutaneously and this may be due, not only to their size relative to the capillary pore size, it may also be due to their slow diffusion through the interstitium because of their size. It is understood that macromolecules can possess discrete domains having a hydrophobic and/or hydrophilic nature. In contrast, small molecules which are hydrophilic are generally well absorbed when administered subcutaneously and it is possible that no enhanced absorption profile would be seen upon injection into the dermis compared to absorption following subcutaneous administration. Reference to hydrophobic substances herein is intended to mean low molecular weight substances, for example substances with molecular weights less than 1000 Daltons, which have a water solubility which is low to substantially insoluble.

IV-like pharmacokinetics is accomplished by administering drugs into the dermal compartment in intimate contact with the capillary microvasculature and lymphatic microvasculature. It should be understood that the terms microcapillaries or capillary beds refer to either vascular or lymphatic drainage pathways within the dermal area. While not intending to be bound by any theoretical mechanism of action, it is believed that the rapid absorption observed upon administration into the dermis is achieved as a result of the rich plexuses of blood and lymphatic vessels in the dermis. However, the presence of blood and lymphatic plexuses in the dermis would not by itself be expected to produce an enhanced absorption of macromolecules. This is because capillary endothelium is normally of low permeability or impermeable to macromolecules such as proteins, polysaccharides, nucleic acid polymers, substance having polymers attached such as pegylated proteins and the like. Such macromolecules have a molecular weight of at least 1000 Daltons or of a higher molecular weight of at least, 2000 Daltons, at least 4000 Daltons, at least 10,000 Daltons or even higher. Furthermore, a relatively slow lymphatic drainage from the interstitium into the vascular compartment would also not be expected to produce a rapid increase in plasma concentration upon placement of a pharmaceutical substance into the dermis.

One possible explanation for the unexpected enhanced absorption reported herein is that upon injection of substances so that they readily reach the papillary dermis an increase in blood flow and capillary permeability results. For example, it is known that a pinprick insertion to a depth of 3 mm produces an increase in blood flow and this has been postulated to be independent of pain stimulus and due to tissue release of histamine (Arildsson et al, Microvascular Res. 59:122-130, 2000). This is consistent with the observation that an acute inflammatory response elicited in response to skin injury produces a transient increase in blood flow and capillary permeability (see Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172,1971). At the same time, the injection into the intradermal layer would be expected to increase interstitial pressure. It is known that increasing interstitial pressure from values, beyond the “normal range” of about −7 to about +2 mmHg distends lymphatic vessels and increases lymph flow (Skobe et al., J. Investig. DermatoL Symp. Proc. 5:14-19, 2000). Thus, the increased interstitial pressure elicited by injection into the intradermal layer is believed to elicit increased lymph flow and increased absorption of substances injected into the dermis.

Directly targeting the dermal space as taught by the invention provides more rapid onset of effects of substances, including drugs, diagnostic substances and therapeutic substances. The inventors have found that substances can be rapidly absorbed and systemically distributed via controlled ID administration that selectively accesses the dermal vascular and lymphatic microcapillaries, thus the substances may exert their beneficial effects more rapidly than SC administration. This effect has special significance for drugs requiring rapid onset, such as insulin (to decrease blood glucose), pain relief substances such as those used for managing breakthrough cancer pains, migraine relief drugs, or emergency rescue drugs (e.g., adrenaline or anti-venom). Natural hormones are also released in a pulsatile fashion with a rapid onset burst followed by a rapid clearance and can benefit from the methods of the invention. Examples of hormones that can benefit from the methods of the invention include insulin, released in response to biological stimulus, for example high glucose levels; female reproductive hormones, which are released at time intervals in a pulsatile fashion; and Human growth hormone; released in normal patients in a pulsatile fashion during sleep. Although not intending to be bound by a particular mechanism of action, the methods of the invention allow better therapy regimens by mimicking the natural body rhythms with synthetic drug compounds. Likewise, it may better facilitate some current therapies such as blood glucose control via insulin delivery. Many current attempts at preparing “closed, loop” insulin pumps are hindered by the delay period between administering the insulin and waiting for the biological effect to occur. This makes it difficult to ascertain in real-time whether sufficient insulin has been given, without overtitrating and risking hypoglycemia. The more rapid PK/PD of ID delivery eliminates much of this type of problem.

Delivering substances to the intradermal compartment in accordance with the methods of the invention results in a more rapid systemic distribution and offset of substances, including drugs, diagnostic substances and therapeutic substances. Therefore, the present invention is particularly useful for delivery of many hormones that are secreted in a pulsatile fashion. Many side effects are associated with having continuous circulating levels of substances administered. A pertinent example is female reproductive hormones that actually have the opposite effect, i.e., cause infertility, when continuously present in the blood. Likewise, continuous and elevated levels of insulin are suspected to down regulate insulin receptors both in quantity and sensitivity.

Substances administered in accordance with the methods of the invention achieve higher bioavailabilities. This effect has been most dramatic for ID administration of high molecular weight substances, especially proteins, peptides, and polysaccharides. Although not intending to be bound by a particular mechanism of action, higher bioavailibilities of substances delivered to the intradermal compartment allows equivalent biological effects while using less active agent. Therefore, higher bioavailability may allow reduced overall dosing and thus decrease the patient's side effects associated with higher dosing regimens. Reduction of the effective therapeutic dose of a substance results in a direct economic benefit to the drug manufacturer and perhaps consumer, especially for expensive protein therapeutics and diagnostics.

Substances administered in accordance with the methods of the invention achieve higher maximum concentrations of the substance compared to the concentration achieved using conventional modes of administration. The inventors have found that substances administered ID are absorbed more rapidly, resulting in a higher initial concentrations. This effect is most beneficial for substances whose efficacy is related to maximal concentration. Although not intending to be bound by a particular mechanism of action, the more rapid onset allows a higher C_{Max} value to be reached with lesser amounts of the substance. Therefore, the effective dose of the substance can be reduced, providing an economic benefit, as well as a physiological benefit since lesser amounts of the substance has to be cleared by the body.

Substances administered in accordance with the methods of the invention do not alter the pharmacodynamic mechanism or biological response mechanism. Substances administered to the intradermal compartment in accordance with the methods of the invention still exert their effects by the same biological pathways that are intrinsic to other delivery means. Any pharmacodynamic changes are related only to the difference patterns of appearance, disappearance, and drug or diagnostic agent concentrations present in the biological system.

Another benefit of the invention is removal of the physical or kinetic barriers invoked when drugs passes through and becomes trapped in cutaneous tissue compartments prior to systemic absorption. Elimination of such barriers leads to an extremely broad applicability to various drug classes. Many drugs administered subcutaneously exert this depot effect—that is, the drug is slowly released from the SC space, in which it is trapped, as the rate determining step prior to systemic absorption, due to affinity for or slow diffusion through the fatty adipose tissue. This depot effect results in a lower Cₘₐₓ, and longer Tₘₐₓ, compared to ID, and can result in high inter-individual variability of absorption. This effect is also pertinent for comparison to transdermal delivery methods including passive patch technology, with or without permeation enhances, ionotophoretic technology, sonopheresis, or stratum corneum ablation or disruptive methods. Transdermal patch technology relies on drug partitioning through the highly impermeable stratum comeum and epidermal barriers. Few drugs except highly lipophilic compounds can breach this barrier, and those that do, often exhibit extended offset kinetics due to tissue saturation and entrappment of the drugs. Active transdermal means, while often faster than passive transfer means, are still restricted to compound classes that can be moved by charge repulsion or other electronic or electrostatic means, or carried passively through the transient pores caused by cavitation of the tissue during application of sound waves. The stratum corneum and epidermis still provide effective means for inhibiting this transport. Stratum corneum removal by thermal or laser ablation, abrasive means or otherwise, still lacks a driving force to facilitate penetration or uptake of drugs. Direct ID administration by mechanical means overcomes the kinetic barrier properties of skin, and is not limited by the pharmaceutical or physicochemical properties of the drug or its formulation excipients.

Another benefit of the invention is highly controllable dosing regimens. The applicants have determined that ID infusion studies have demonstrated dosing profiles that are highly controllable and predictable due to the rapid onset and offset kinetics of drugs or diagnostic agents delivered by this route. This allows almost absolute control over the desired dosing regimen when ID delivery is coupled with ‘a fluid control means or other control system to regulate metering of the drug or diagnostic agent into the body. This single benefit alone is one of the principal goals of most drug or diagnostic agent delivery methods. Bolus ID substance administration as defined previously results in kinetics most similar to IV injection and is most desirable for pain relieving compounds, mealtime insulin, rescue drugs, erectile dysfunction compounds, or other drugs that require rapid onset. Also included would be combinations of substances capable-of-acting alone or synergistically. Extending the ID administration duration via infusion can effectively mimic SC uptake parameters, but with better predictability. This profile is particularly good for substances such as growth hormones, or analgesics. Longer duration infusion, typically at lower infusion rates can result in continuous low basal levels of drugs that is desired for anticoagulants, basal insulin, and chronic pain therapy. These kinetic profiles can be combined in multiple fashion to exhibit almost any kinetic profile desired. An example would be to pulsatile delivery of fertility hormone (LHRH) for pregnancy induction, which requires intermittent peaks every 90 minutes with total clearance between pulses. Other examples would be rapid peak onset of drugs for migraine relief, followed by lower levels for pain prophylaxis.

Another benefit of the invention is reduced degradation of drugs and diagnostic agents and/or undesirable immunogenic activity. Transdermal methods using chemical enhancers or iontophoresis, or sonophoresis or electroporation or thermal poration require that a drug pass through the viable epidermal layer, which has high metabolic and immunogenic activity. Metabolic conversion of substances in the epidermis or sequestration by immunoglobulins reduces the amount of drug available for absorption. The ID administration circumvents this problem by placing the drug directly in the dermis, thus bypassing the epidermis entirely. Although not intending to be bound by a particular mechanism of action, the rapid trafficking of the substance from the skin into the blood circulation via microcapillary or lymphatic uptake reduces residence time of the substance in the skin, thus reducing the time in which antigen presenting cells can take up the substance and induce an immune response to the substance. Although not intending to be bound by a particular mechanism of action, the intradermal administration of a soluble protein substance without added adjuvants, excipients or depot-forming agents may induce a state of immune tolerance whereby the undesired immune response (e.g., induction of neutralizing antibodies) is reduced or eliminated.

These and other benefits of the invention are achieved by directly targeting absorption by the papillary dermis and by controlled delivery of drugs, diagnostic agents, and other substances to the dermal space of skin. The inventors have found that by specifically targeting the intradermal space and controlling the rate and pattern of delivery, the pharmacokinetics exhibited by specific drugs can be unexpectedly improved, and can in many situations be varied with resulting clinical advantage. Such pharmacokinetics cannot be as readily obtained or controlled by other parenteral administration routes, except by IV access.

The methods of the present invention not only provide improved pharmacokinetics over conventional delivery methods, but also provide additional benefits including a reduction in unwanted immune response caused by the administration of a substance, such as an inadvertent immuno-toxic effect against the active ingredients of the substance. As used herein, the term “unwanted immune response” means the natural immune response of the subject receiving a substance of this invention, where the substance is not intended to provoke such response when administered. Examples of unwanted immune responses that may be prevented using the methods of the invention include, but are not limited to, IgE-mediated hypersensitivity, with the risk of local and/or systemic anaphylactic reaction as described, for instance, after parenteral injection of insulin or heparin and many other drugs based on having a protein or a polysaccharide as the active ingredient; antibody-mediated cytotoxic hypersensitivity as well as immune complex mediated hypersensitivity that cause systemic adverse events, such as kidney and/or liver and/or microvascular alteration due to the deposition of circulating immune complexes; cell-mediated hypersensitivity with the risk of inducing delayed type reaction at the injection site and immune neutralization of the active ingredient, or any systemic adverse event, such as thrombocytopenia induced by heparin treatment. (copy expanded paragraph from above)

Substances delivered to the intradermal compartment of a subject's skin in accordance with the methods of the invention have reduced immunogenicity relative to other compartments including IM and SC. As used herein, the term “immunogenicity” refers to the property of a substance (e.g., foreign objects, microorganisms, drugs, antigens, proteins, peptides, polypeptides, nucleic acids, DNA, RNA, etc.) being able to evoke an immune response within an organism. Immunogenicity depends partly upon the size of the substance in question and partly upon how unlike the host molecules is the substance. Highly conserved proteins tend to have rather low immunogenicity.

The present invention thus provides methods for therapeutic treatment by delivery of a drug or other substance, preferably a therapeutic substance to a human or animal subject by directly targeting the intradermal space, where the drug or substance is administered to the intradermal space through one or more dermal-access means incorporated within the device. Substances delivered according to the methods of the invention have been found to have improved clinical and therapeutic utility over conventional delivery methods including IM and SC, including but not limited to improved PK and reduction of immunogenicity and an undesired immune response.

The invention encompasses a method for directly delivering a substance into an intradermal space within mammalian skin comprising administering the substance through at least one small gauge hollow needle having an outlet with an exposed height between 0 and 1 mm, said outlet being inserted into the skin to a depth of between 0.3 mm and 2 mm, such that delivery of the substance occurs at a depth between 0.3 mm and 2 mm. In a specific embodiment injecting the substance comprises inserting the needle to a depth which delivers the substance at least about 0.3 mm below the surface to no more than about 2 mm below the surface. The invention encompasses administering a substance by inserting the needle into the skin to a depth of at least about 0.3 mm and no more than about 2 mm. The invention encompasses administration of substances at a rate between 1 nL/min. and 200 mL/min. In most preferred embodiments, the needle(s) are inserted substantially perpendicularly to the skin.

In some embodiments, the delivered substance has improved pharmacokinetics compared to pharmacokinetics after subcutaneous injection. The improved pharmacokinetics includes but is not limited to an increased bioavailability of the substance, a decrease in Tₘₐₓ, an increase in Cₘₐₓ, a decrease in T_{lag}, an enhanced absorption rate or a combination thereof.

In a specific embodiment, the administration of a substance in accordance with the methods of the invention is through at least one small gauge hollow needle. In another specific embodiment, the administration of a substance in accordance with the methods of the invention uses a needle that has an outlet with an exposed height between 0 and 1 mm.

The invention encompasses administration of peptides, proteins, nucleic acids and hormones. In specific embodiments, the protein or peptide may be selected from the group consisting of insulin, granulocyte stimulating factor and PTH. Substances administered in accordance with the methods of the invention may be hydrophobic or hydrophilic. Substances administered in accordance with the methods of the invention may have a molecular weight of less than or greater than 1000 daltons.

The invention encompasses a method of administering a pharmaceutical substance comprising injecting or infusing the substance intradermally through one or more microneedles having a length and outlet suitable for selectively delivering the substance into the dermis to obtain absorption of the substance in the dermis. In preferred embodiments, absorption of the substance in the dermis produces improved systemic pharmacokinetics compared to subcutaneous administration. The improved pharmacokinetics includes but is not limited to an increased bioavailability, decreased Tₘₐₓ, an increase in Cₘₐₓ, a decrease in T_{lag}, or an enhanced absorption rate or a combination thereof. The length of the microneedle used in this embodiment may be from about 0.5 mm to about 1.7 mm. This embodiment encompasses a microneedle which is a 30 to 34 gauge needle. The microneedle may have an outlet of from 0 to 1 mm. In preferred embodiments, the microneedle is configured in a delivery device which positions the microneedle perpendicular to skin surface. In some embodiments, the microneedle is contained in an array of microneedles which may comprise of 3 microneedles or 6 microneedles.

The invention encompasses a microneedle for intradermal injection of a pharmaceutical substance, wherein the microneedle has a length and outlet selected for its suitability for specifically delivering the substance into the dermis. The length of the microneedle may be from about 0.5 mm to about 1.7 mm. The microneedle may be a 30 to 34 gauge needle. The microneedle may have an outlet of from 0 to 1 mm. Preferably the microneedle is configured in a delivery device which positions the microneedle perpendicular to skin surface. In some embodiments, the microneedle is contained in an array of microneedles which may comprise of 3 microneedles or 6 microneedles.

The invention encompasses a method for delivering a bioactive substance to a subject comprising contacting the skin of the subject with a device having a dermal-access means for accurately targeting the dermal space of the subject with an efficacious amount of the bioactive substance. In some embodiments, the pharmacokinetics of the bioactive substance is improved relative to the pharmacokinetics of the substance when administered subcutaneously. The improved pharmacokinetics may be an increase in bioavailability, a decrease in Tₘₐₓ, an increase in Cₘₐₓ, a decrease in T_{lag}, or an enhanced absorption rate or any combination thereof. The device used in this embodiment may have a fluid driving means including a syringe, infusion pump, piezoelectric pump, electromotive pump, electromagnetic pump, or Belleville spring. The dermal access means may comprise one or more hollow microcannula having a length of from about 0.5 mm to about 1.7 mm. The dermal access means may comprise one or more hollow microcannula having an outlet with an exposed height between 0 and 1 mm.

In other embodiments, the invention encompasses a method for delivering a bioactive substance to a subject comprising contacting the skin of a subject with a device having a dermal-access means for accurately targeting the dermal space of the subject with an efficacious amount of the bioactive substance at a rate of 1 nL/min. to 200 mL/min. In this specific embodiment, the onset pharmacokinetics of the bioactive substance is substantially improved relative to subcutaneous injection. Alternatively other PK parameters are improved including for example an increase in bioavailability, a decreased Tₘₐₓ, an increase in Cₘₐₓ, a decreased T_{lag}, or an enhanced absorption rate. The dermal access means in accordance with this embodiment may have one or more hollow microcannula that inserts into the skin of said subject to a depth of from about 0.5 to about 2.0 mm. The dermal access means may have one or more hollow microcannula having an outlet with an exposed height between 0 and 1 mm.

### 5.1 Intradermal Administration Methods

The present invention encompasses methods for intradermal delivery of substances described and exemplified herein to the intradermal compartment of a subject's skin, preferably by selectively and specifically targeting the intradermal compartment without passing through it. In a most preferred embodiment, the intradermal compartment is targeted directly. Once a formulation containing the substance to be delivered is prepared, the formulation is typically transferred to an injection device for intradermal compartment delivery, e.g., a syringe. Delivery of the formulations of the invention in accordance with the methods of the invention provides an improved therapeutic and clinical efficacy of the substance over conventional modes of delivery including IM and SC by specifically and selectively, preferably directly targeting the intradermal compartment. The intradermal delivery methods of the invention provide benefits and improvements over including but not limited to improved pharmacokinetics, reduced immunogenicity, and reduction of undesired immune response. The methods of the invention are particularly effective for administrations of therapeutic substances to which the induction of an immune response would not be beneficial to the therapeutic effect of the substance to be delivered. The methods of the present invention result in an improved pharmacokinetics such as an improved absorption uptake within the intradermal compartment. The formulations of the invention may be delivered to the intradermal space as a bolus or by infusion.

The formulations of the invention are administered using any of the intradermal devices and methods disclosed in U.S. patent application Ser. No. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The intradermal methods of administration comprise microneedle-based injection and infusion systems or any other means to accurately target the intradermal space. The intrademal methods of administration encompass not only microdevice-based injection means, but other delivery methods such as needless or needle-free ballistic injection of fluids or powders into the intradermal space, Mantoux-type intradermal injection, enhanced iontophoresis through microdevices, and direct deposition of fluid, solids, or other dosing forms into the skin.

In a specific embodiment, the formulations of the invention are administered to an intradermal compartment of a subject's skin using an intradermal Mantoux type injection, see, e.g., Flynn et al., 1994, Chest 106: 1463-5, which is incorporated herein by reference in its entirety. In a specific embodiment, the formulation of the invention is delivered to the intradermal compartment of a subject's skin using the following exemplary method. The formulation is drawn up into a syringe, e.g., a 1 mL latex free syringe with a 20 gauge needle; after the syringe is loaded it is replaced with a 30 gauge needle for intradermal administration. The skin of the subject, e.g., mouse, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention encompasses the use of conventional injection needles, catheters or microneedles of all known types, employed singularly or in multiple needle arrays. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

The invention encompasses ballistic fluid injection devices, powder jet delivery devices, piezoelectric, electromotive, electromagnetic assisted delivery devices, gas-assisted delivery devices, which directly penetrate the skin to directly deliver the formulations of the invention to the targeted location within the dermal space.

The actual method by which the formulations of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the methods of the invention preferably targets the formulations of the invention to a depth of at least at least 0.5 mm up to a depth of no more than 2.5 mm, more preferably no more than 2.0 mm, and most preferably no more than 1.7 mm. In some embodiments, the formulations are delivered at a targeted depth just under the stratum comeum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhan's cells in the dermal space of human skin, delivery would need to encompass, at least, in part, the epidermal tissue depth typically ranging from about 0.025 mm to about 0.2 mm in humans.

The formulations delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

The invention also encompasses varying the targeted depth of delivery of formulations of the invention. The targeted depth of delivery of formulations may be controlled manually by the practitioner, or with or without the assistance of an indicator to indicate when the desired depth is reached. Preferably however, the devices used in accordance with the invention have structural means for controlling skin penetration to the desired depth within the intradermal space. The targeted depth of delivery may be varied using any of the methods described in U.S. patent application Ser. No. 09/417,671, filed on Oct. 14, 1999; Ser. No. 09/606,909, filed on Jun. 29, 2000; Ser. No. 09/893,746, filed on Jun. 29, 2001; Ser. No. 10/028,989, filed on Dec. 28, 2001; Ser. No. 10/028,988, filed on Dec. 28, 2001; or International Publication No.'s EP 10922 444, published Apr. 18, 2001; WO 01/02178, published Jan. 10, 2002; and WO 02/02179, published Jan. 10, 2002; all of which are incorporated herein by reference in their entirety.

The benefits of the invention including improved pharmacokinetics and reduced immunogenicity are best realized by accurate direct targeting of the dermal capillary beds. This is accomplished, for example, by using microneedle systems of less than about 250 micron outer diameter, and less than 2 mm exposed length. Such systems can be constructed using known methods of various materials including steel, silicon, ceramic, and other metals, plastic, polymers, sugars, biological and or biodegradable materials, and/or combinations thereof.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion. That is, at a greater depth a needle outlet with a greater exposed height will still seal efficiently where as an outlet with the same exposed height will not seal efficiently when placed in a shallower depth within the intradermal space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height it is understood that a single needle may have more than one opening or outlets suitable for delivery of substances to the dermal space.

It has also been found that by controlling the pressure of injection or infusion may avoid the high backpressure exerted during ID administration. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out-of the-skin. The appropriate delivery rates and volumes to obtain these effects for a selected substance may be determined experimentally using only ordinary skill. Increased spacing between multiple needles allows broader fluid distribution and increased rates of delivery or larger fluid volumes. In addition, it has been found that ID infusion or injection often produces higher initial plasma levels of drug than conventional SC administration, particularly for drugs that are susceptible to in vivo degradation or clearance or for compounds that have an affinity to the SC adipose tissue or for macromolecules that diffuse slowly through the SC matrix. This may, in many cases, allow for smaller doses of the substance to be administered via the ID route.

The administration methods useful for carrying out the invention include both bolus and infusion delivery of drugs and other substances to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver a substance the dermal-access means is placed adjacent to the skin of a subject providing directly targeted access within the intradermal space and the substance or substances are delivered or administered into the intradermal space where they can act locally or be absorbed by the bloodstream and be distributed systematically. The dermal-access means may be connected to a reservoir containing the substance or substances to be delivered. The form of the substance or substances to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. Delivery from the reservoir into the intradermal space may occur either passively, without application of the external pressure or other driving means to the substance or substances to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the substance may be variably controlled by the pressure-generating means. As a result, the substance enters the intradermal space and is absorbed in an amount and at a rate sufficient to produce a clinically efficacious result.

As used herein, the term “clinically efficacious result” is meant a clinically useful biological response including both diagnostically and therapeutically useful responses, resulting from administration of a substance or substances. For example, diagnostic testing or prevention or treatment of a disease or condition is a clinically efficacious result. Such clinically efficacious results include diagnostic results such as the measurement of glomerular filtration pressure following injection of inulin, the diagnosis of adrenocortical function in children following injection of ACTH, the causing of the gallbladder to contract and evacuate bile upon injection of cholecystokinin and the like as well as therapeutic results, such as clinically adequate control of blood sugar levels upon injection of insulin, clinically adequate management of hormone deficiency following hormone injection such as parathyroid hormone or growth hormone, clinically adequate treatment of toxicity upon injection of an antitoxin and the like.

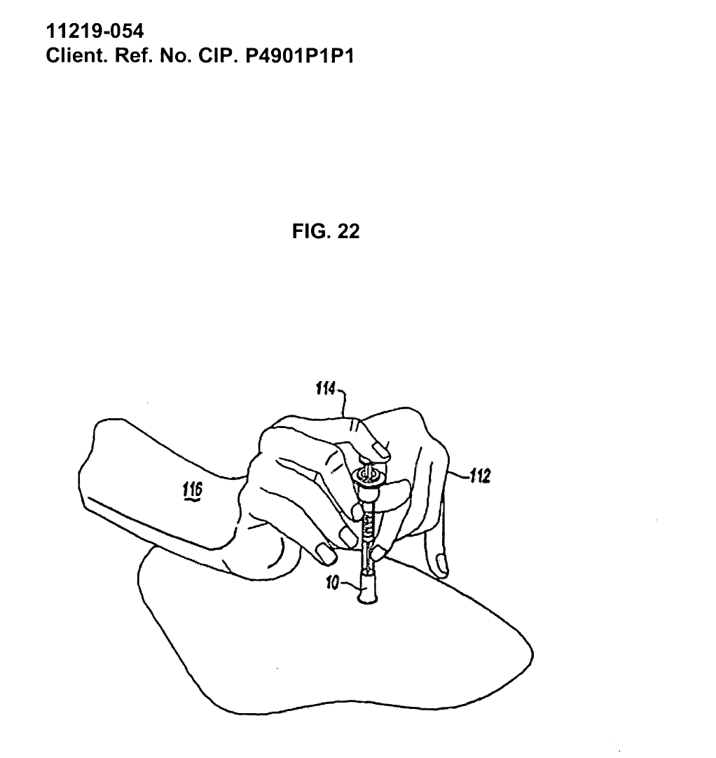

Exemplary modes of intradermal injections using exemplary devices are shown in FIGS. 22-25. Having described the intradermal delivery device including the needle assembly 20 and drug container 60 supra, its operation and use in practicing the methods of the present invention for intradermally injecting substances is described below.

Prior to inserting the needle cannula 24, an injection site upon the skin of the animal is selected and cleaned. Subsequent to selecting and cleaning the site, the forward end 40 of the needle cannula 24 is inserted into the skin of the animal at an angle of generally 90 degrees until the skin engaging surface 42 contacts the skin. The skin engaging surface 42 prevents the needle cannula 42 from passing through the dermis layer of the skin and injecting the substance into the subcutaneous layer.

While the needle cannula 42 is inserted into the skin, the substance is intradermally injected. The substance may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the substance. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus includes selecting a syringe for injection having an inside diameter of sufficient width to generate a force sufficient to overcome the back pressure of the dermis layer when the substance is expelled from the syringe to make the injection.

In addition, since intradermal injections are typically carried out with small volumes of the substance to be injected, e.g., on the order of no more than 0.5 mL, and preferably around 0.1 mL, a syringe barrel 60 with a small inside diameter is preferred to minimize dead space which could result in wasted substance captured between the stopper 70 and the shoulder of the syringe after the injection is completed. Also, because of the small volumes of substance, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the substance within the barrel of the syringe.

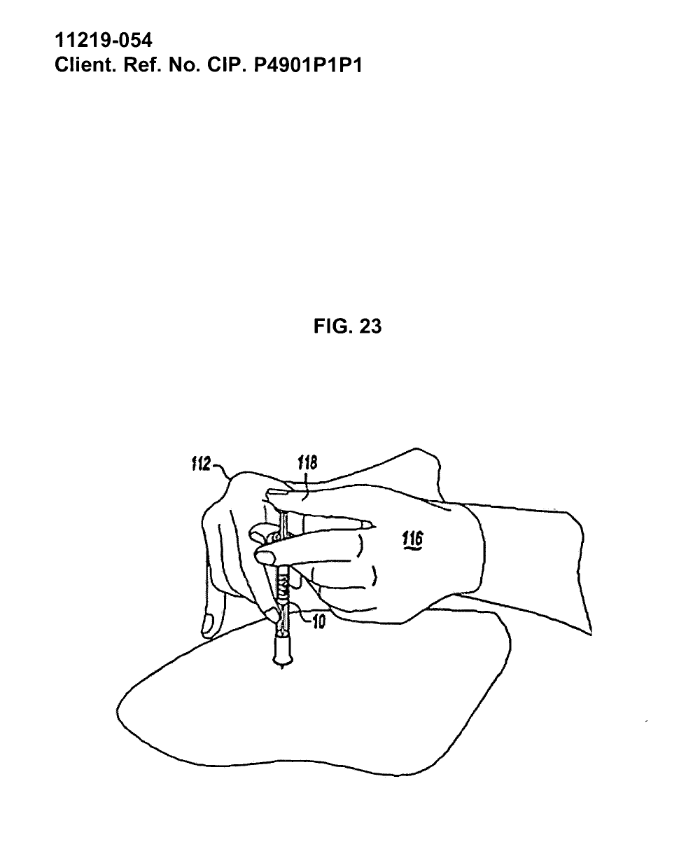

As shown in FIG. 22, the syringe 60 may be grasped with a first hand 112 and the plunger 66 depressed with the forefinger 114 of a second hand 116. Alternatively, as shown in FIG. 23 the plunger 66 may be depressed by the thumb 118 of the second hand 116 while the syringe 60 is held by the first hand. In each of these variations, the skin of the animal is depressed, and stretched by the skin engaging surface 42 on the limiter 26. The skin is contacted by neither the first hand 112 nor the second hand 116.

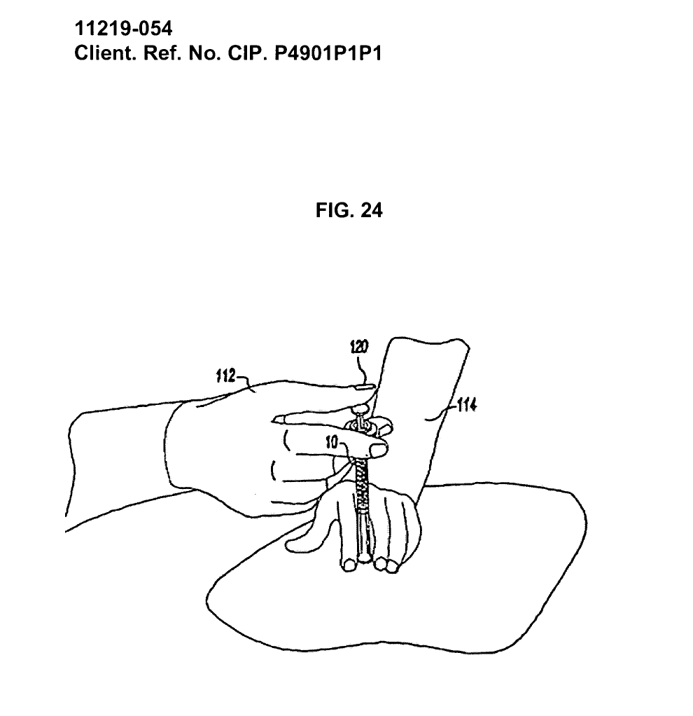

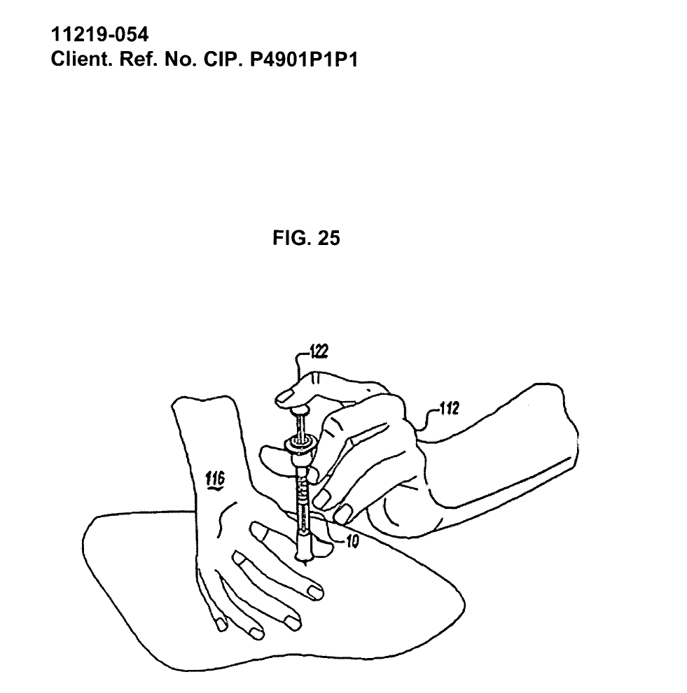

An additional variation has proven effective for administering the intradermal injection of the present invention. This variation includes gripping the syringe 60 with the same hand that is used to depress the plunger 66. FIG. 24 shows the syringe 60 being gripped with the first hand 112 while the plunger is simultaneously depressed with the thumb 120 of the first hand 112. This variation includes stretching the skin with the second hand 114 while the injection is being made. Alternatively, as shown in FIG. 25, the grip is reversed and the plunger is depressed by the forefinger 122 of the first hand 112 while the skin is being stretched by the second hand 116. However, it is believed that this manual stretching of the skin is unnecessary and merely represents a variation out of habit from using the standard technique.

In each of the variations described above, the needle cannula 24 is inserted only about 1.5 mm into the skin of the animal. Subsequent to administering the injection, the needle cannula 24 is withdrawn from the skin and the syringe 60 and needle assembly 20 are disposed of in an appropriate manner. Each of the variations were utilized in clinical trials to determine the effectiveness of both the needle assembly 20 and the present method of administering the intradermal injection.

In a specific embodiment the invention encompasses a method of making an injection into the skin of an animal comprising the following steps: (1) providing a drug delivery device, which includes a needle cannula having a forward needle tip such that the needle cannula is in fluid communication with a substance contained in the drug delivery device and includes a limiter portion surrounding the needle cannula and the limiter portion includes a skin engaging surface, so that the needle tip of the needle cannula extends from the limiter portion beyond the skin engaging surface a distance equal to approximately 0.5 mm to approximately 3.0 mm and the needle cannula has a fixed angle of orientation relative to a plane of the skin engaging surface of the limiter portion; (2) inserting the needle tip into the skin of an animal and engaging the surface of the skin with the skin engaging surface of the limiter portion such that the skin engaging surface of the limiter portion limits penetration of the needle tip into the dermis layer of the skin of the animal; and (3) expelling the substance from the drug delivery device through the needle tip into the skin of the animal. In some embodiments, the angle of the orientation of the needle cannula relative to a plane of the skin engaging surface is 90 degrees.

### 5.1.1 Dosing and Frequency of Administration

The amount of the formulation of the invention which will be effective in the prevention, treatment, management, or amelioration of a disorder (e.g., a proliferative disorder, an inflammatory disorder), or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, as well as age, body, weight, response, and the past medical history of the subject. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a formulation of the invention include milligram or microgram amounts of the therapeutic substance per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). In some embodiments, the dosage levels are not adjusted based on the subject's body weight, e.g., a constant dose is administered for all subjects. In yet other embodiments, the dosing regimen will consist of a dose escalation, whereby the subject is initially treated with a low dose of the therapeutic substance that is followed by increased dosages until a plateau dosage level is achieved. In a specific embodiment, the invention encompasses methods of administration with no dose escalation. In another specific embodiment, the methods of the invention comprise administration of a formulation described or exemplified herein comprising the following dose escalation schedule: during weeks 1-2, administration of a dose of 8.8 μg, during weeks 3-4, administration of a dose of 22 μg, during week 5 to end of therapy, administration of a dose of 44 μg. In yet another specific embodiment, the methods of the invention comprise administration of a formulation described or exemplified herein comprising the following dose regimen: during weeks 1-2, administration of a dose of 62.5 μg, during weeks 3-4, administration of a dose of 125 μg, during weeks 5-6, administration of a a dose of 187.5 μg, during week 7 to end of therapy, administration of a a dose of 250 μg. In other embodiments the invention encompasses administration of a dose of at least 250 μg every other day for at least 2 weeks, at least one month, at least two months, at least 4 months, or up to the life time of the subject.

In general, the recommended daily dose range of a substance of the invention for the conditions described herein lie within the range of from about 0.01 mg to about 500 mg per day, given as a single once-a-day dose. In some embodiments, the dose may be divided doses throughout a day. In one embodiment, the daily dose is administered twice daily in equally divided doses. The invention encompasses, a daily dose range from about 5 μg to about 500 μg per day, more specifically, between about 10 μg and about 200 μg per day. Preferably, the invention encompasses a dose range from about 22 μg to about 250 μg. In managing a subject in accordance with the methods of the invention, the therapy may be initiated at a lower dose, perhaps about 1 μg to about 25 μg, and increased if necessary up to about 200 μg to about 1000 mg per day as either a single dose or divided doses, depending on the subject's global response, as measured for example by improvements in clinical symptoms of a disorder for which the therapy is intended. In some embodiments, the dosage may be administered not daily, but rather multiple times per week (e.g., every other day, 2-times per week, 3-times per week, etc.). It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the formulations of the invention are also encompassed by the above described dosage amounts and dose frequency schedules. Further, when a subject is administered multiple dosages of a formulation of the invention, not all of the dosages need be the same. For example, the dosage administered to the subject may be increased to improve the prophylactic or therapeutic effect of the formulation or it may be decreased to reduce one or more side effects that a particular subject is experiencing.

In a specific embodiment, the dosage of the formulation of the invention administered to prevent, treat, manage, or ameliorate a disorder, or one or more symptoms thereof in a subject is at least 0.05 μg/kg, preferably at least 0.1 μg/kg, at least 0.5 μg/kg, 1 mg/kg, or more of a subject's body weight. In another embodiment, the dosage of the formulation of the invention administered to prevent, treat, manage, or ameliorate a disorder or one or more symptoms thereof in a subject is a unit dose of at least 10 μg, at least 20 μg, at least 30 μg, at least 50 μg, at least 100 μg, or at least 500 μg. In some embodiments, the formulations of the invention are administered chronically e.g., administered for over a time period of at least 2 months, at least 4 months, at least 6 months, or up to the life time of the subject. In a specific embodiment, the dosage of the formulation of the invention administered to prevent, treat, manage, or ameliorate a disorder or one or more symptoms thereof in a subject is is not escalated during treatment. In another specific embodiment, the dosage of the formulation of the invention administered to prevent, treat, manage, or ameliorate a disorder or one or more symptoms thereof in a subject comprises the following dose escalation: during weeks 1-2, administration of a dose of 8.8 μg, during weeks 3-4, administration of a dose of 22 μg, during week 5 to end of therapy, administration of a dose of 44 μg. In yet another specific embodiment, the dosage of the formulation of the invention administered to prevent, treat, manage, or ameliorate a disorder or one or more symptoms thereof in a subject comprises the following dose regimen: during weeks 1-2, administration of a dose of 62.5 μg, during weeks 3-4, administration of a dose of 125 μg, during weeks 5-6, administration of a dose of 187.5 μg, during week 7 to end of therapy, administration of a dose of 250 μg. In other the embodiments, the dosage of the formulation of the invention administered to prevent, treat, manage, or ameliorate a disorder or one or more symptoms thereof in a subject comprises administration of a dose of at least 250 μg every other day for at least 2 weeks, at least one month, at least two months, at least 4 months, or up to the life time of the subject.

In a specific embodiment, the invention provides a method of preventing, treating, managing, or ameliorating a disorder, or one or more symptoms thereof, said method comprising: (a) administering to a subject in need thereof a dose of about 22 μg to 250 μg of one or more formulations of the invention); and (b) administering one or more subsequent doses to said subject when there is a detectable antibody titer in the subject. In another embodiment, the invention provides a method of preventing, treating, managing, or ameliorating a disorder, or one or more symptoms thereof, said method comprising: (a) administering to a subject in need thereof one or more doses of about 22 μg to 250 μg of one or more formulations of the invention; (b) monitoring the mean antibody titer using for example an ELISA assay in said subject after the administration of a certain number of doses; and (c) administering a subsequent dose of the formulation(s) of the invention when there is a detectable antibody titer in the subject.

### 5.2 Devices for Intradermal Delivery

The present invention encompasses any device for accurately and selectively targeting the intradermal compartment of a subject's skin. The nature of the device used is not critical as long as it penetrates the skin of the subject to the targeted depth within the intradermal compartment without passing through it. Preferably the device penetrates the skin at a depth of at least about 0.5 mm, preferably at least 1.0 mm up to a depth of no more than 3.0 mm. Although not intending to be bound by a particular mechanism of action, placement of the substance predominantly at greater depths and/or into the lower portion of the reticular dermis may result in the substance being slowly absorbed in the less vascular reticular dermis or in the SC compartment, either of which would result in reduced absorption of the substance.

The invention compasses drug delivery devices and needle assemblies disclosed in U.S. Pat. No. 6,494,865 and U.S. patent application Ser. Nos. 10/357,502 and 10/337,413 (filed on Feb. 4, 2003 and Jan. 7, 2003, respectively), PCT application 2004/02783 filed Jan. 30, 2004; U.S. patent application Ser. No. 10/916,649 filed Aug. 12, 2004 all of which are incorporated herein by reference in their entireties.

The dermal-access means of the inventions are devices designed for targeted intradermal delivery and encompasses microneedle-based injection and infusion systems or any other means to accurately target the intradermal space. The invention also encompasses other delivery methods such as needless or needle-free ballistic injection of fluids or powders into the ID space, Mantoux-type ID injection, enhanced iontophoresis through microdevices, and direct deposition of fluid, solids, or other dosing forms into the skin.

The dermal-access means used for ID administration according to the invention is not critical as long as it penetrates the skin of a subject to the desired targeted depth within the intradermal space without passing through it. In most cases, the device will penetrate the skin and to a depth of about 0.5-2 mm. The dermal-access means may comprise conventional injection needles, catheters or microneedles of all known types, employed singularly or in multiple needle arrays. The dermal-access means may comprise needleless devices including ballistic injection devices. The terms “needle” and “needles” as used herein are intended to encompass all such needle-like structures. The term microneedles as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. Dermal-access means also include ballistic fluid injection devices, powder jet delivery devices, piezoelectric, electromotive, electromagnetic assisted delivery devices, gas-assisted delivery devices, of which directly penetrate the skin to provide access for delivery or directly deliver substances to the targeted location within the dermal space.

The invention encompasses delivering a substance to the intradermal compartment of a subject's skin using a device comprising at least one needle, preferably a microneedle. In some embodiments, the devices of the invention are needleless. Preferably the needle has a length sufficient to penetrate the intradermal space and an outlet at a depth within the intradermal space so that the substance is delivered and deposited therein. In general the needle is no longer than about 2 mm long, preferably 300 μm to 2 mm; most preferably 500 μm to 1 mm. The needle outlet is typically at a depth of about 250 μm to 2 mm when the needle is inserted in the skin, preferably at a depth of 750 μm to 1.5 and most preferably at a depth of about 1 mm.

Microneedles used in the methods of the invention are also very sharp and of a very small gauge such as 30 or 34 G, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The junctional delivery devices of the invention may also incorporate reservoirs to contain the substance prior to delivery or pumps or other means for delivering the drug or other substance under pressure. Alternatively, the junctional delivery devices may be linked externally to such additional components.

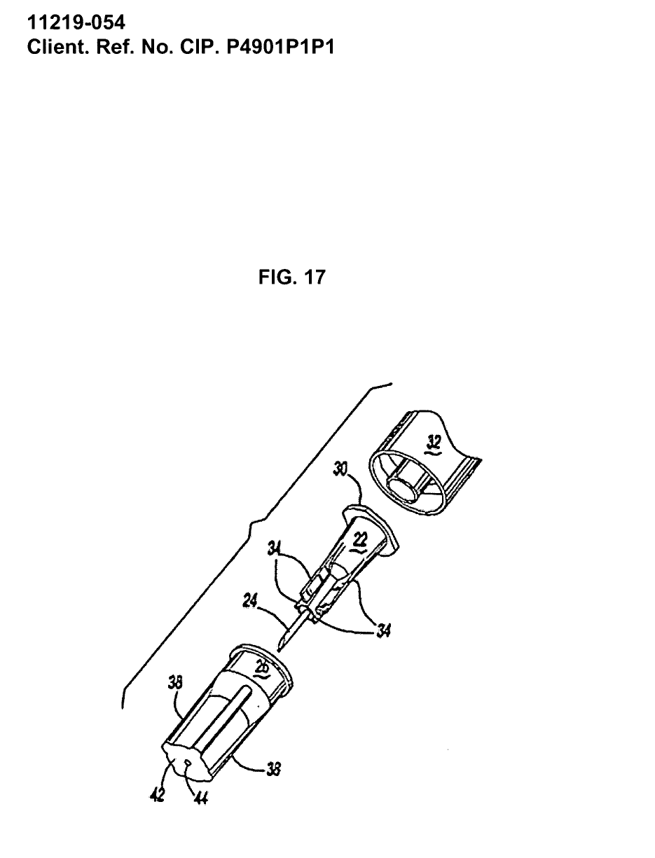

FIGS. 17-19 of the drawings illustrate an example of a drug delivery device which can be used to practice the methods of the present invention for making intradermal injections (FIGS. 22-25). The device 10 illustrated in FIGS. 17-19 includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety.

The needle assembly 20 includes a hub 22 that supports a needle cannula 24. The limiter 26 receives at least a portion of the hub 22 so that the limiter 26 generally surrounds the needle cannula 24 as best seen in FIG. 17.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order. `

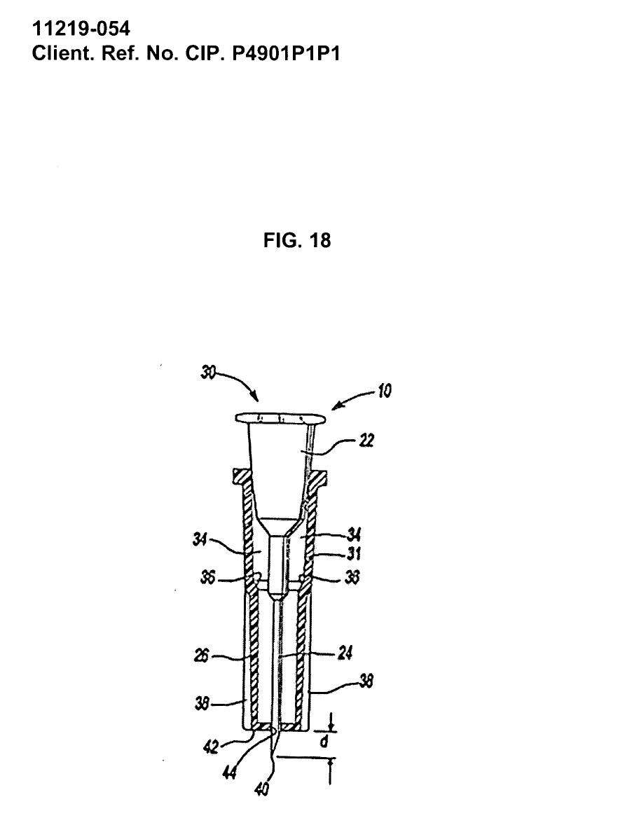

As can be best seen in FIG. 18, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the animal's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

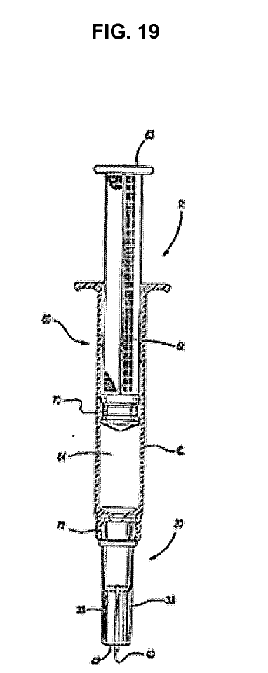

It is important to note that although FIGS. 17 and 18 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 18 and 19. Additionally, it is possible to adhesively or otherwise secure the hub 22 to the limiter 26 in the position illustrated in FIG. 18 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 19 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 19, such needle assembly designed is readily adaptable to a wide variety of conventional syringe styles.

This invention provides an intradermal needle injector that is adaptable to be used with a variety of syringe types. Therefore, this invention provides the significant advantage of facilitating manufacture and assembly of intradermal needles on a mass production scale in an economical fashion.The devices for use in the invention may comprise conventional injection needles, catheters or microneedles of all known types, employed singularly or in multiple needle arrays. The devices may comprise powder jet delivery devices, piezoelectric, electromotive, electromagnetic assisted delivery devices, gas-assisted delivery devices, of which directly penetrate the skin to provide access for delivery or directly deliver substances to the targeted location within the skin.

The length of microneedles are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The devices for use in the methods of the invention may also incorporate reservoirs to contain the substance prior to delivery or pumps or other means for delivering the drug or other substance under pressure.

The devices for use in accordance with the methods of the invention may comprise any means for controlling rates and/or pressures of delivery using pumping mechanism including but not limited to syringe pumps (e.g., Harvard Syringe Pumps), infusion pumps (e.g., microinfusion pumps), mechanical springs (e.g., coil springs, belleville springs, washers), elastomeric membrane, gas pressure devices, piezoelectric devices, electromotive based devices, or electromagnetic based devices, or any other device known in the art for controlling rates of delivery. Additionally any of the devices and methods disclosed in U.S. Pat. Nos. 5,957,895 and 6,074,369 may be used in accordance with the instant invention (the specified patents are incorporated herein by reference in their entireties).

The invention further encompasses varying the targeted depth of delivery. By varying the targeted depth of delivery of substances by the dermal-access means, pharmacokinetic and pharmacodynamic (PK/PD) behavior of the drug or substance can be tailored to the desired clinical application most appropriate for a particular patient's condition. The targeted depth of delivery of substances by the dermal-access means may be controlled manually by the practitioner, or with or without the assistance of indicator means to indicate when the desired depth is reached. Preferably however, the device has structural means for controlling skin penetration to the desired depth within the intradermal space. This is most typically accomplished by means of a widened area or hub associated with the shaft of the dermal-access means that may take the form of a backing structure or platform to-which the needles are attached.

The length of microneedles as dermal-access means are easily varied during the fabrication process and are routinely produced in less than 2 mm length. Microneedles are also very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

The present invention improves the clinical utility of ID delivery of drugs, diagnostic agents, and other substances to humans or animals. The methods employ dermal-access means (for example a small gauge needle, especially microneedles), to directly target the intradermal space and to deliver substances to the intradermal space as a bolus or by infusion. It has been discovered that the placement of the dermal-access means within the dermis provides for efficacious delivery and pharmacokinetic control of active substances. The dermal-access means is so designed as to prevent leakage of the substance from the skin and improve adsorption within the intradermal space. The pharmacokinetics of hormone drugs delivered according to the methods of the invention have been found to be vastly different to the pharmacokinetics of conventional SC delivery of the drug, indicating that ID administration according to the methods of the invention will provide improved clinical results. Delivery devices that place the dermal-access means at an appropriate depth in the intradermal space and control the volume and rate of fluid delivery provide accurate delivery of the substance to the desired location without leakage.

### 5.3 Formulations for Intradermal Delivery

The invention encompasses formulations comprising one or more substances for intradermal delivery. In some embodiments, the formulations containing a substance of the invention comprise a therapeutically or prophylactically effective amount of the substance. Substances for use in the methods of the invention are preferably formulated so that they do not induce an immune response when delivered to the intradermal compartment. Preferably formulations of the invention contain no additives, including but not limited to excipients and adjuvants, which enhance the antigenicity or immunogenicity of the substances when delivered to the intradermal compartment. Although not intending to be bound by any mechanism of action substances delivered in accordance with the methods of the invention based, in part, on their formulations, do not enhance presentation and/or availability of the substance to the immune cells of the intradermal compartment, rather they are rapidly systemically distributed thus bypassing the immune system.

As used herein, and unless otherwise specified, a “therapeutically effective amount” refers to an amount of a substance of the present invention or other active ingredient sufficient to provide a therapeutic benefit in the treatment or management of a disease or to delay or minimize symptoms associated with the disease. Further, a therapeutically effective amount with respect to a substance of the invention means that amount alone, or in combination with other therapies, provides a therapeutic benefit in the treatment or management of the disease. Used in connection with an amount of a substance of the present invention, the term can encompass an amount that improves overall therapy, reduces or avoids symptoms or causes of disease, or enhances the therapeutic efficacy of or synergies with another therapeutic agent.

As used herein, and unless otherwise specified, a “prophylactically effective amount” refers to an amount of a substance of the invention or other active ingredient sufficient to result in the prevention, recurrence or spread of the disease. A prophylactically effective amount may refer to the amount sufficient to prevent initial disease, the recurrence or spread of the disease or the occurrence of the disease in a patient, including but not limited to those predisposed to the disease. A prophylactically effective amount may also refer to the amount that provides a prophylactic benefit in the prevention of the disease. Further, a prophylactically effective amount with respect to a substance of the invention means that amount alone, or in combination with other agents, provides a prophylactic benefit in the prevention of the disease. Used in connection with an amount of a substance of the invention, the term can encompass an amount that improves overall prophylaxis or enhances the prophylactic efficacy of or synergies with another prophylactic agent.

In some embodiments, the formulations of the invention comprise one or more other additives. Additives that may be used in the formulations containing a substance of the invention include for example, wetting agents, emulsifying agents, or pH buffering agents. The formulations containing a substance of the invention may contain one or more other excipients such as saccharides and polyols. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the formulations of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety). In preferred embodiments, the formulations of the invention (e.g., the interferon beta formulations) comprise albumin at a concentration of at least 2 mg, at least 4 mg, at least 15 mg, or at least 20 mg.

The formulations of the invention can be a solid, such as a lyophilized powder suitable for reconstitution, a liquid solution, a suspension, a tablet, a pill, a capsule, a sustained release formulation, or a powder.

The formulations containing a substance of the present invention can be prepared using any accepted methods of preparation known in the art. The specific method of preparation depends on the specific substance to be administered, and such variations are within the ordinary skill in the art.

The invention encompasses methods of administering solution and particulate forms of a substance of the invention and mixture thereof, including fast-acting, intermediate-acting, and long-acting formulations that may be obtained from any substances. The formulations used in the methods and formulations of the invention may be a mixture of one or more formulations that contain a substance of the present invention.

The substances in the formulations may be in different physical association states, for example, monomeric or dimeric states. The chemical state of the substance may be modified by standard recombinant DNA technology to produce the substance of different chemical formulas in different association states. Alternatively solution parameters, such as pH, may be altered to result in formulations of the substance in different association states. Other chemical and/or genetic modifications of the substances of the invention are also encompassed by the instant invention.

Using the methods of the invention lower doses of substances are required to achieve a similar therapeutic efficacy as conventional methods of administration. The substances delivered in accordance with the methods of the invention have improved therapeutic efficacy compared to the same substances delivered by conventional methods.

In the case that biological molecules are to be administered, such molecules may be from different animal species including, limited but not to, swine, bovine, ovine, equine, etc.

The invention encompasses formulations in which a substance of the present invention is in a particulate form, i.e., is not fully dissolved in solution. In some embodiments, at least 30%, at least 50%, at least 75% of the substance is in particulate form. Although not intending to be bound by a particular mode of action, formulations of the invention in which the substance is in particulate form have at least one agent which facilitates the precipitation of the substance. Precipitating agents that may be employed in the formulations of the invention may be proteinacious, e.g., protamine, a cationic polymer, or non-proteinacious, e.g., zinc or other metals or polymers.

The form of a substance to be delivered or administered include solutions thereof in pharmaceutically acceptable diluents or solvents, emulsions, suspensions, gels, particulates such as micro- and nanoparticles, either suspended or dispersed, as well as in-situ forming vehicles of the same. The formulations containing a substance of the invention may be in any form suitable for junctional delivery. In one embodiment, the junctional formulation of the invention is in the form of a flowable, injectible medium, i.e., a low viscosity formulation that may be injected in a syringe. The flowable injectible medium may be a liquid. Alternatively the flowable injectible medium is a liquid in which particulate material is suspended, such that the medium retains its fluidity to be injectible and syringable, e.g., can be administered in a syringe.

The formulations of the present invention can be prepared as unit dosage forms. A unit dosage per vial may contain 0.1 to 1 mL of the formulation. In some embodiments, a unit dosage form of the junctional formulations of the invention may contain about 50 μL to 100 μL, 50 μL to 200 μL, 50 μL to 500 μL or 50 μL to 1 μL of the formulation. If necessary, these preparations can be adjusted to a desired concentration by adding a sterile diluent to each vial. The volumes of the formulations administered in accordance with the methods of the invention are not administered in volumes whereby the junctional layer might become overloaded leading to partitioning to one or more other compartments, such as the subcutaneous compartment. However, the volume of the formulation when using the junctional administration of the present invention is much less critical that when other conventional administration methods are used. Without being bound by a particular theory, it is believed that the junctional injection is much more receptive to a larger volume bolus due to the flexibility and high deformability of the junctional connective tissue. Therefore, using the methods of the present invention, injection volume of about 0.5 mL or greater, more specifically about 1.0 mL or greater, may be administered into the junctional layer

### 5.3.1 Substances

Substances delivered in accordance with the methods of the invention have an improved clinical utility and therapeutic efficacy relative to other drug delivery methods including intramuscular, and subcutaneous delivery. Substances that can be delivered to the intradermal compartment of a subject's skin in accordance with the present invention include pharmaceutically or biologically active substances include diagnostic agents, drugs, and other substances which provide therapeutic or health benefits such as for example nutraceuticals. The invention encompasses the administration of any protein, particularly a therapeutic protein, and all salts, polymorphs, analogs, derivatives, fragments, mimetics, and peptides thereof. Substances that are particularly suited for the methods of the invention are those which can benefit from a reduced risk of unwanted immune response as encountered by conventional modes of administration including IM and SC. The methods of the invention are particularly useful for chronic administration of substances, e.g., therapeutic proteins which typically result in an unwanted immune response upon chronic administration by other routes. Preferred substances for use in the methods of the invention are, cytokines (e.g., interferons, including interferon alpha and beta) chemokines, immunomodulatory agents and therapeutic proteins. (see above re: antibodies, etc)

In preferred embodiments, the invention encompasses administration of cytokines. Any cytokine used in the art for therapeutic or experimental purposes are encompassed within the present invention. Cytokines are growth factors, secreted primarily from leukocytes and stimulate both humoral and cellular immune responses as well as the activation of phagocytic cells. The invention encompasses administration of lymphokines, (secreted from lymphocytes) and monokines (secreted by monocytes and macrophages). In some embodiments, the invention encompasses administration of lymphokines such as interleukins, including but not limited to IL1-α, IL1-β, IL-2, IL-3, IL-4, IL-5, IL-6, IL-7, IL-8, IL-9, IL-10, IL-11, IL-12, IL-13.

The invention encompasses administration of immunomodulatory agents in accordance with the methods of the invention. As used herein, the term “immunomodulatory agent” and variations thereof including, but not limited to, immunomodulatory agents, refer to an agent that modulates a host's immune system. In certain embodiments, an immunomodulatory agent is an immunosuppressant agent. In certain other embodiments, an immunomodulatory agent is an immunostimulatory agent. Immunomodatory agents include, but are not limited to, small molecules, peptides, polypeptides, fusion proteins, antibodies, inorganic molecules, mimetic agents, and organic molecules. Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators.

Diagnostic substances useful in the present invention include macromolecular substances such as, for example, insulin, ACTH (e.g., corticotropin injection), luteinizing hormone-releasing hormone (e.g., Gonadorelin Hydrochloride), growth hormone-releasing hormone (e.g., Sermorelin Acetate), cholecystokinin (e.g., Sincalide), parathyroid hormone and :fragments thereof (e.g., Teriparatide Acetate), thyroid releasing hormone and analogs thereof (e.g., protirelin), secretin and the like.

Therapeutic substances which can be used with the present invention include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons (including interferon-α and interferon-β; “IFN”), Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-α, and TNF-α antagonist,

The invention also encompasses administering monoclonal antibodies, peglyated antibodies, pegylated proteins or any proteins modified with hydrophilic or hydrophobic polymers or additional functional groups, fusion proteins, single chain antibody fragments or the same with any combination of attached proteins, macromolecules, or additional functional groups thereof. Examples of therapeutic antibodies that can be used in methods of the invention include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAn™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Other substances that may be used in the methods and formulations of the invention included major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In specific embodiments, where the substance to be administered is a protein or a fragment thereof, any recombinant DNA technology known to one skilled in the art may be used to produce the protein. A variety of host-expression vector systems may be utilized to express the proteins or fragments for use in the methods of the invention. Such host-expression systems are well known and provide the necessary means by which a protein of interest may be produced and subsequently purified. Examples of host-expression vector systems that may be used in accordance with the invention are: bacterial cells (e.g., E. coli, B. subtilis) transformed with recombinant bacteriophage DNA, plasmid DNA or cosmid DNA expression vectors containing nucleic acid coding sequence encoding a protein, yeast cells (e.g., Saccharomyces, Pichia) transformed with a recombinant yeast expression vector containing the protein of interest coding sequence; insect cells infected with a recombinant virus expression vector (e.g., baculovirus) containing the protein of interest coding sequence; plant cells infected with a recombinant virus expression vector (e.g., cauliflower mosaic virus, CaMV; tobacco mosaic virus, TMV) or transformed with a recombinant plasmid expression vector (e.g., Ti plasmid) containing the protein of interest coding sequence; or mammalian cells (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected. Such vectors include but are not limited to the E. coli expression vector pUR278 (Ruther et al., 1983, EMBO J. 2:1791), in which the protein of interest coding sequence can be ligated into the vector in-frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors can also be used to express foreign polypeptides as fusion proteins with glutathione S-transferase (GST). In general, such fusion proteins are soluble and can easily be purified from lysed cells by adsorption and binding to a column comprising of glutathione-agarose beads followed by elution in the presence of free glutathione. The pGEX vectors are designed to include, e.g., thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The protein of interest coding sequence can be cloned individually into non-essential regions (for example the polyhedrin gene) of the virus and placed under control of an AcNPV promoter (for example the polyhedrin promoter). Successful insertion of the protein of interest coding sequence will result in inactivation of the polyhedrin gene and production of non-occluded recombinant virus (i.e., virus lacking the proteinaceous coat coded for by the polyhedrin gene). These recombinant viruses can be used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems can be utilized. In cases where an adenovirus is used as an expression vector, the protein of interest coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the protein of interest in infected hosts (see, e.g., Logan & Shenk, 1984, Proc. Natl. Acad. Sci. USA 81:3655). Specific initiation signals may also be required for efficient translation of inserted the protein of interest coding sequences. These signals include the ATG initiation codon and adjacent sequences. In cases where an entire protein of interest, including its own initiation codon and adjacent sequences, is inserted into the appropriate expression vector, no additional translational control signals may be needed. However, in cases where only a portion of the protein of interest coding sequence is inserted, exogenous translational control signals, including, if necessary, the ATG initiation codon, must be provided. These exogenous translational control signals and initiation codons can be of a variety of origins, both natural and synthetic. Furthermore, the initiation codon must be in phase with the reading frame of the desired coding sequence to ensure correct translation of the entire insert. The efficiency of expression may be enhanced by the inclusion of appropriate transcription enhancer elements, transcription terminators, etc. (see Bittner et al., 1987, Methods in Enzymol. 153: 516).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, COS, MDCK, 293, 3T3, WI38, and in particular, breast cancer cell lines such as, for example, BT483, Hs578T, HTB26, BT20 and T47D, and normal mammary gland cell lines such as, for example, CRL7030 and Hs578Bst.

### 5.3.2 Interferons

In particularly preferred embodiments, the invention encompasses administration of interferons to the intradermal compartment of a subject's skin. Interferons (IFN) as used herein carry their ordinary meaning in the art and refer to cytokines characterized by antiviral, antiproliferative, and immunomodulatory activities. The interferons are divided into the type I and type II classes. Interferon beta belongs to the class of type I interferons, which also includes interferons alpha, tau and omega, whereas interferon gamma. is the only known member of the distinct type II class. The invention encompasses administration of both classes of interferons.

The invention encompasses natural, recombinant, synthetic variants of interferons known to one skilled in the art (for a review see, Cell Mol. Life Sci. 54:1203-1206, 1998, which is incorporated herein by reference in its entirety). Methods for producing interferons are experimentally routine and encompassed within the invention, e.g., E. coli expression, expression in CHO cells. In a specific embodiment, interferons are provided consisting of or comprising a fragment of an interferon consisting or comprising of at least ten contiguous amino acids, at least 20, at least 30, at least 40, at least 50 contiguous amino acids, up to at least 166 amino acids. The invention encompasses recombinant interferons that are glycosylated at one or more sites. In some embodiments, the recombinant interferons for use in the formulations of the invention are not glycosylated. The amino acid sequence of human IFN-β was reported by Taniguchi, (1980, Gene 10:11-15), and in EP 83069, EP 41313 and U.S. Pat. No. 4,686,191, all of which are incorporated herein by reference in their entireties. Crystal structures have been reported for human and murine IFN-β, respectively (Proc. Natl. Acad. Sci. USA 94:11813-11818, 1997; J. Mol. Biol. 253:187-207, 1995; each of which is incorporated herein by reference in its entirety).

The invention encompasses recombinantly engineered variants of IFN-β known to one skilled in the art such as those disclosed in WO 9525170, WO 9848018, U.S. Pat. Nos. 5,545,723, 4,914,033, EP 260350, U.S. Pat. Nos. 4,588,585, 4,769,233, 6,531,122 Stewart et al., 1987, DNA 6(2):119-128, Runkel et al., 1998, J. Biol. Chem. 273(14): 8003-8008; all of which are incorporated herein by reference in their entireties. Interferon-β may be produced using any method known to one skilled in the art for example by expression of in CHO cells, see, e.g., U.S. Pat. Nos. 4,966,843, 5,376,567 and 5,795,779, all of which are incorporated herein by reference in their entireties.

The invention encompasses human commercial preparations of IFN-β including but not limited to Betaseron® (Berlex, also termed IFN-β1b, which is non-glycosylated, produced using recombinant bacterial cells, has a deletion of the N-terminal metionine residue and the C17S mutation); Avonex® (Biogen, also termed IFN-β1a, and is identical to the natural protein); and Rebif® (Serono, also termed IFN-β1a, which is glycosylated, produced using recombinant mammalian cells). The table below summarizes the commercially accepted routes and dosages of administration for commercial preparations of human IFN-β. The methods of the invention have an improved clinical utility and therapeutic efficacy for administration of interferons relative to the conventional modes of delivery, IM and SC. The enhanced efficacy of the interferon formulations of the invention results in a therapeutically effective response with lower doses and/or lower frequencies of administration of interferons as conventionally used.

TABLE 1
INTERFERON BETA COMMERCIAL PREPARATIONS

| DRUG | ROUTE | DOSE | FREQUENCY |
|---|---|---|---|
| Rebif ® | SC | 44 μg | 3×/week |
| Biogen ® | IM | 30 μg | 1×/week |
| Betaseron ® | SC | 250 μg | Every other day |

The invention additionally encompasses commercially available preparation of IFN-β from any other species, including rats, e.g., those available from Cell Sciences Inc. (Canton, Mass.); see, also Ruuls et al., 1996, J. Immunol. 157: 5721-31; which is incorporated herein by reference in its entirety.

The invention encompasses chronic administration of IFN-β formulations in accordance with the methods of the invention. In some embodiments, IFN-β formulations are administered for at least 2 months, at least 4 months, at least 6 months, up to the life time of the subject. In some embodiments, the dosing regimen comprises administration of the IFN-β formulations at least once a weak, at least twice a week, at least 3 times a week, or up to once a day. In a specific embodiment, the dosing regimen of an IFN-β formulation may comprise the following: a dose of at least 0.129 μg/kg for weeks 1-2, at dose of 0.324 μg/kg for weeks 3-4, and a dose of 0.647 μg/kg for weeks 5 to the end of the treatment.

The dosage of the IFN-β formulation of the invention administered to prevent, treat, manage, or ameliorate a Multiple sclerosis (MS), or one or more symptoms thereof in a subject is at least 0.05 μg/kg, preferably at least 0.1 μg/kg, at least 0.5 μg/kg, 1 mg/kg, or more of a subject's body weight. In another embodiment, the dosage of the formulation of the invention administered to prevent, treat, manage, or ameliorate a disorder or one or more symptoms thereof in a subject is a unit dose of at least 10 μg, at least 20 μg, at least 30 μg, at least 50 μg, at least 100 μg, or at least 500 μg. In some embodiments, the formulations of the invention are administered chronically for at least 2 months, at least 4 months, at least 6 months, or up to the life time of the subject.

### 5.4 Prophylactic and Therapeutic Uses

The present invention encompasses administering a therapeutically or prophylactically effective amount of one or more of the substances disclosed herein to an animal, preferably a mammal, and most preferably a human, for preventing, treating, or ameliorating one or more symptoms associated with a disease, disorder, or infection wherein the substance is delivered to the intradermal compartment of the subject's skin. As used herein, the terms “disorder” and “disease” are used interchangeably to refer to a condition in a subject. Substances of the invention may be provided in pharmaceutically acceptable formulations as known in the art or as described herein. As detailed below, the substances of the invention can be used in methods of treating, preventing, managing, or ameliorating one or more symptoms associated with cancer, viral infections, e.g., hepatitis, autoimmune disease, inflammatory disorders and multiple sclerosis. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders. As used herein, the term “cancer” refers to a neoplasm or tumor resulting from abnormal uncontrolled growth of cells. In some embodiments, cancer refers to a benign tumor, which has remained localized. In other embodiments, cancer refers to a malignant tumor, which has invaded and destroyed neighboring body structures and spread to distant sites. In some embodiments, the cancer is associated with a specific cancer antigen

The substances of the invention may also be advantageously utilized in combination with other therapeutic agents known in the art for the treatment or prevention of a cancer, autoimmune disease, inflammatory disorders or infectious diseases. In a specific embodiment, molecules of the invention may be used in combination with monoclonal or chimeric antibodies, lymphokines, or hematopoietic growth factors (such as, e.g., IL-2, IL-3 and IL-7), which, for example, serve to increase the number or activity of effector cells which interact with the molecules and, increase immune response. The molecules of the invention may also be advantageously utilized in combination with one or more drugs used to treat a disease, disorder, or infection such as, for example anti-cancer agents, anti-inflammatory agents or anti-viral agents.

### 5.4.1 Target Diseases

### 5.4.1.1 Multiple Sclerosis (MS)

In some embodiments, the methods of the invention encompass delivering a formulation comprising a therapeutically effective amount of IFN-β to the intradermal compartment of a subject's skin for the treatment and/or prevention of Multiple sclerosis (MS). MS is a crippling disease that affects over 250,000 Americans. MS is characterized by neuron deterioration in the central nervous system with the associated lose of the insulating myelin sheath from around the axons of the nerve cells. This loss of myelin results in loss of electrical insulation and the “short-circuiting” of the electrical pathways mediated by the affected nerves and progressive neurological impairment. MS usually affects young adults in what should be the healthiest, most productive years of their lives and affects women more often than men. The symptoms of MS include pain and tingling in the arms and legs; localized and generalized numbness, muscle spasm and weakness; bowel and bladder dysfunction; difficulty with balance when walking or standing; and fatigue. In most cases, people afflicted with MS lose the ability to stand and/or walk entirely. Optic neuritis may occur episodically throughout the course of the disease. The symptoms are exacerbated by physical fatigue or emotional stress. Approximately half the people with this disease have relapsing-remitting MS in which there are unpredictable attacks where the clinical symptoms become worse (exacerbation) which are separated by periods of remission where the symptoms stabilize or diminish. The other half have chronic progressive MS without periods of remission.

In a specific embodiment, the invention encompasses administering a formulation comprising a therapeutically effective amount of IFN-β comprising delivering the formulation to the intradermal compartment of the subject's skin, for the treatment, prevention, management or amelioration of one or more symptoms associated with multiple sclerosis (MS). Preferably, the methods of the invention are therapeutically more effective than conventional methods of IFN-β therapy for MS. Although not intending to be bound by a particular mechanism of action, the therapeutic utility of IFN-β may be due, in part, to its inhibitory effects on the proliferation of leukocytes and antigen presentation, its modulation of the profile of cytokine production towards an anti-inflammatory phenotype, and a reduction of T-cell migration by inhibiting the activity of T-cell matrix metalloproteases.

The therapeutic methods of the invention are more effective than conventional modes of MS therapy by reducing the frequency and severity of exacerbations, extending the time between relapses, reducing the immunogenicity of chronic IFN-β administration. The therapeutic methods of the invention are preferably longer lasting and more cost effective than conventional modes of MS therapy. More preferably, the methods of the invention do not result in any adverse effects at the site of injection which is in contrast to conventional modes of IFN-β delivery, such as IM and SC. The methods of the invention have reduced to minimal side effects associated with the use of current modes of delivery of IFN-β, including injection site reactions, fever, chills, myalgias, arthralgias, and other flu-like symptoms (Clin. Therapeutics, 19:883-893, 1997, which is incorporated herein by reference in its entirety). In addition, 6-40% of patients develop neutralizing antibodies to IFN-β (Int. Arch. Allergy Immunol. 118:368-371, 1999). It has been shown that development of IFN-β neutralizing antibodies decreases the biological response to IFN-β, and cause a trend towards decreased treatment efficacy (Neurol. 50:1266-1272, 1998). Neutralizing antibodies will likely also impede the therapeutic utility of IFN-β in connection with treatment of other diseases (Immunol. Immuther. 39:263-268, 1994). The methods of the invention reduce or do not alter the production of neutralizing antibodies associated with conventional modes of delivery of IFN-β, while concomitantly providing improved pharmacokinetics of the drug.

The invention encompasses use of the IFN-β formulations of the invention for the treatment of osteosarcoma, basal cell carcinoma, cervical dysplasia, glioma, acute myeloid leukemia, multiple myeloma, Hodgkin's disease, breast carcinoma, melanoma, and viral infections such as papilloma virus, viral hepatitis, herpes genitalis, herpes zoster, herpetic keratitis, herpes simplex, viral encephalitis, cytomegalovirus pneumonia, and rhinovirus.

### 5.4.1.2 Cancer

The invention encompasses methods and formulations for treatment and/or prevention of cancer or metastasis in a subject comprising delivering a therapeutically or prophylactically effective amount of a therapeutic substance to the intradermal compartment of a subject's skin. In some embodiments, substances of the invention may be administered in combination with a therapeutically or prophylactically effective amount of one or additional therapeutic agents known to those skilled in the art for the treatment and/or prevention of cancer.

For example, but not by way of limitation, cancers associated with the following cancer antigens may be treated or prevented by the methods and compositions of the invention: KS ¼ pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl. Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81(6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Niittelman et al., 1990, J. Clin. Invest. 86:2136-2144)), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erthrocytes and primary endoderm, I(Ma) found in gastric adencarcinomas, M18 and M39 found in breast epithelium, SSEA-1 found in myeloid cells, VEP8, VEP9, Myl, VIM-D5, and D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma, CO-514 (blood group Le^{a}) found in adenocarcinoma, NS-10 found in adenocarcinomas, CO-43 (blood group Le^{b}), G49, EGF receptor, (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, GM₂, OFA-2, GD₂, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

Cancers and related disorders that can be treated or prevented by methods and compositions of the present invention include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypemephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Infonned Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, prostate, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

In one embodiment, angiogenesis inhibitors may be administered in combination with the substances of the invention. Angiogenesis inhibitors that can be used in the methods and compositions of the invention include but are not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Anti-cancer agents that can be used in combination with the substances of the invention in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol,9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltiterxed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin.

### 5.4.1.3 Autommune and Inflammatory Diseases

The invention encompasses methods and formulations for treatment and/or prevention of an autoimmune or inflammatory disease in a subject comprising delivering a therapeutically or prophylactically effective amount of a therapeutic substance to the intradermal compartment of a subject's skin. The invention also provides methods for preventing, treating, or managing one or more symptoms associated with an inflammatory disorder in a subject further comprising, administering to said subject a therapeutically or prophylactically effective amount of one or more anti-inflammatory agents. The invention also provides methods for preventing, treating, or managing one or more symptoms associated with an autoimmune disease further comprising, administering to said subject a therapeutically or prophylactically effective amount of one or more immunomodulatory agents. Anon-limiting examples of anti-inflammatory agents and immunomodulatory agents is provided below.

Examples of autoimmune disorders that may be treated by administering the substances of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. As described supra, some autoimmune disorders are associated with an inflammatory condition. Thus, there is overlap between what is considered an autoimmune disorder and an inflammatory disorder. Therefore, some autoimmune disorders may also be characterized as inflammatory disorders. Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections.

The method of the present invention provides methods of treatment for autoimmune diseases and inflammatory diseases comprising administration of the formulations of the present invention in conjunction with other treatment agents. Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steriods (e.g., cortisol, cortison, Fludrocortisone, Prednisone, Prednisolone, Triamcinolone, Betamethasone, Dexamethasone), mycophenolate mofetil, rapamycin (sirolimus), mizoribine, deoxyspergualin, brequinar, malononitriloamindes (e.g., leflunamide), T cell receptor modulators, and cytokine receptor modulators.

Steroids that may be used in the methods and compositions of the invention include but are not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide.

Anti-inflammatory agents have exhibited success in treatment of inflammatory and autoimmune disorders and are now a common and a standard treatment for such disorders. Any anti-inflammatory agent well-known to one of skill in the art can be used in the methods of the invention. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

### 5.4.1.4 Infectious Diseases

The invention encompasses methods and formulations for treatment and/or prevention of an infectious or viral disease in a subject comprising delivering a therapeutically or prophylcatically effective amount of a therapeutic substance to the intradermal compartment of a subject's skin.

Viral diseases that can be treated or prevented using the molecules of the invention in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be treated or prevented using the molecules of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases that can be treated or prevented using the molecules of the invention in conjunction with the methods of the present invention, that are caused by protozoa include, but are not limited to, leishmania, kokzidioa, trypanosoma or malaria.

Parasitic diseases that can be treated or prevented using the molecules of the invention in conjunction with the methods of the present invention, that are caused by parasites include, but are not limited to, chlamydia and rickettsia.

In some embodiments, substances of the invention may be administered in combination with a therapeutically or prophylactically effective amount of one or additional therapeutic agents known to those skilled in the art for the treatment and/or prevention of an infectious disease. The invention contemplates the use of the substances of the invention in combination with antibiotics known to those skilled in the art for the treatment and or prevention of an infectious disease. Antibiotics that can be used in combination with the molecules of the invention include, but are not limited to, macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)),aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In certain embodiments, substances of the invention can be administered in combination with a therapeutically or prophylactically effective amount of one or more antifungal agents. Antifungal agents that can be used in combination with the molecules of the invention include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

In some embodiments, substances of the invention can be administered in combination with a therapeutically or prophylactically effective amount of one or more anti-viral agent. Useful anti-viral agents that can be used in combination with the molecules of the invention include, but are not limited to, protease inhibitors, nucleoside reverse transcriptase inhibitors, non-nucleoside reverse transcriptase inhibitors and nucleoside analogs. Examples of antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, pleconaril.

### 5.4.2 Comination Therapy

The invention further encompasses administering the substances of the invention in combination with other therapies known to those skilled in the art for the treatment or prevention of a disease or disorder. For example for the treatment of cancer, the invention encompasses other therapies including but not limited to, current standard and experimental chemotherapies, hormonal therapies, biological therapies, immunotherapies, radiation therapies, or surgery. In some embodiments, the substances of the invention may be administered in combination with a therapeutically or prophylactically effective amount of one or more anti-cancer agents, therapeutic antibodies, or other agents known to those skilled in the art for the treatment and/or prevention of a disease. In certain embodiments, one or more substances of the invention is administered to a mammal, preferably a human, concurrently with one or more other therapeutic agents useful for the treatment of cancer. The term “concurrently” is not limited to the administration of prophylactic or therapeutic agents at exactly the same time, but rather it is meant that a substance of the invention and the other agent are administered to a mammal in a sequence and within a time interval such that the substance of the invention can act together with the other agent to provide an increased benefit than if they were administered otherwise. For example, each prophylactic or therapeutic agent (e.g., chemotherapy, radiation therapy, hormonal therapy or biological therapy) may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In other embodiments, the prophylactic or therapeutic agents are administered at about 2 to 4 days apart, at about 4 to 6 days apart, at about 1 week part, at about 1 to 2 weeks apart, or more than 2 weeks apart. In preferred embodiments, the prophylactic or therapeutic agents are administered in a time frame where both agents are still active. One skilled in the art would be able to determine such a time frame by determining the half life of the administered agents.

In certain embodiments, the prophylactic or therapeutic agents of the invention are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic agents are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the therapeutic and prophylactic agents of the invention are administered in metronomic dosing regimens, either by continuous infusion or frequent administration without extended rest periods. Such metronomic administration can involve dosing at constant intervals without rest periods. Typically the therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the therapeutic and prophylactic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled oncologist.

In other embodiments, courses of treatment are administered concurrently to a mammal, i.e., individual doses of the therapeutics are administered separately yet within a time interval such that molecules of the invention can work together with the other agent or agents. For example, one component may be administered one time per week in combination with the other components that may be administered one time every two weeks or one time every three weeks. In other words, the dosing regimens for the therapeutics are carried out concurrently even if the therapeutics are not administered simultaneously or within the same patient visit.

When used in combination with other prophylactic and/or therapeutic agents, the substances of the invention and the prophylactic and/or therapeutic agent can act additively or, more preferably, synergistically. In one embodiment, a substance of the invention is administered concurrently with one or more therapeutic agents in the same pharmaceutical composition. In another embodiment, a substance of the invention is administered concurrently with one or more other therapeutic agents in separate pharmaceutical compositions. In still another embodiment, a substance of the invention is administered prior to or subsequent to administration of another prophylactic or therapeutic agent. The invention contemplates administration of a substance of the invention in combination with other prophylactic or therapeutic agents by the same or different routes of administration, e.g., oral and parenteral. In certain embodiments, when a substance of the invention is administered concurrently with another prophylactic or therapeutic agent that potentially produces adverse side effects-including, but not limited to, toxicity, the prophylactic or therapeutic agent can advantageously be administered at a dose that falls below the threshold that the adverse side effect is elicited.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (57^{th} ed., 2002, which is incorporated herein by reference in its entirety).

### 5.5 Determination of Therapeutic Efficacy of Formulations

The invention encompasses methods for determining the efficacy of formulations of the invention using any standard method known in the art or described herein. Assays for determining the efficacy of the formulations of the invention may be in vitro based assays or in vivo based assays, including animal based assays. In some embodiments, the invention encompasses detecting and/or quantitating a humoral immune response against the formulation of the invention in a sample, e.g., serum or mucosal wash, obtained from a subject who has been administered a formulation of the invention. Preferably, the humoral immune response of the formulation of the invention is compared to the humoral response of the same formulation administered by an alternative route such as IM and/or SC. The methods of the invention encompass measuring isotype specific antibody responses, including but not limited to IgG1, IgG2a, IgG2b or IgG3 using standard methods known in the art, such as an ELISA assay. Although not intending to be bound by a particular mechanism of action, IgG1 specific antibody response suggests a Th2 response, which involves production of antibody to target the antigen, whereas IgG2 response suggests a Th1 response (Ig2a for mice, IgG2b for rats) which are generally cell-mediated responses, indicating the activation of more cells to target the antigen.

Assays for measuring humoral immune response are well known in the art, e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with a formulation of this invention relative to the amount of the antibody in an untreated subject. Preferably, the humoral immune response of a formulation of the invention when delivered in accordance with the methods of the invention to the intradermal compartment, is compared to the humoral immune response when the same formulation is delivered by an alternative route such as IM or SC. ELISA assays can be used to determine total antibody titers in a sample obtained from a subject treated with a composition of the invention. In other embodiments, ELISA assays may be used to determine the level of specific antibody isotypes and antibodies to neutralizing epitopes using methods known in the art.

ELISA based assays comprise preparing an antigen, coating the well of a 96 well microtiter plate with the antigen, adding test and control samples containing antigen specific antibody, adding a detector antibody specific to the antibody in test and control samples that is conjugated to an enzyme (e.g., horseradish peroxidase or alkaline phosphatase) and incubating for a period of time, and detecting the presence of the antigen with a color yielding substrate. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The invention encompasses methods for determining the efficacy of the formulations of the invention by measuring cell-mediated immune responses. Methods for measuring cell-mediated immune responses are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

In cases where the formulation of the invention comprises IFN-β any method known in the art for the detection and/or quantitation of an antibody response against IFN-β is encompassed within the methods of the invention. An exemplary method for determining the IFN-β directed antibody response may comprise the following: sera from an animal, e.g., rat, treated chronically with IFN-β from the same species (e.g., rat IFN-β) are analyzed for total antibody titer against the IFN-β. 96 well plates are coated with the antigen (IFN-β); sample sera are then incubated in the wells; any antibody in the sera will bind to the antigen on the plate; a secondary antibody conjugated with a chromophore is added and will bind to any primary antibody in the well; a developer is then added and the plate read on a spectrophotometer. In a specific embodiment the ELISA assay for measuring antibody response against IFN-β comprises the following: 100 μL of 1 μg/mL of rat IFN-β in coating buffer is added to the wells of a 96 well plate; the plate is incubated at 4° C. overnight; well contents are removed and the wells are blocked with 5% skim milk in PBSt for 2 hours at 37° C.; wells are washed and 100 μL of serum samples are added to the first row of wells; samples are serially diluted across the plate; the plate is incubated for 1 hour at 37° C. and washed; 100 μL of 1:4000 diluted, anti-rat HRP-conjugated antibody is added to each well and the plate incubated for 30-60 minutes at 37° C.; 100 μL of TMB color developer are added to each well; 200 μL of hydrochloric acid reaction stopper is added to each well; the plate is read at 450 nm; the antibody titer is the dilution giving 3 times absorbance of blank serum.

The invention encompasses any method known in the art for the detection and/or quantitation of isotype specific antibody responses against IFN-β. An exemplary method for detection and/or quantitation of isotype specific antibody responses against IFN-β comprises use of an ELISA Quantification Kit, commercially available from Bethyl Laboratories (Bethyl Inc., Montgomery, Tex.). An exemplary ELISA assay comprises the following: coating a 96 well microtiter place (Nunc Maxi-sorb plate) with 1 μg/mL of IFN antigen and incubating overnight at 4° C. (to generate a standard curve wells are coated with 1 μL of capture antibody to 100 μL coating buffer); washing the plates with 50 mM Tris buffered saline, pH 8.0, 0.05% Tween (Sigma #T9039); adding 200 μL of Post Coat/Block solution (50 mM Tris buffered saline, pH 8.0, 1% BSA; Sigma #T6789) to each well and incubating the plate covered for 30 minutes at room temperature. Standards are prepared according to the package insert using the Sample/Conjugate Diluent (0.5 mL of 10% Tween 20; Sigma # P7949) per 100 mL of PostCoat/Block Solution; sample dilutions are made using the Sample/Conjugate Diluent. 100 μL of standard or sample is transferred to assigned wells and incubated for 1 hour at room temperature; conjugate is prepared according to package insert (1:20,000 for IgG1 and 1:40,000 for IgG2b); 100 μL of the conjugate is added per well and incubated for 1 hour at room temperature; 100 μL of TMB per well is added and incubated at room temperature for 30 minutes; 200 μL of 0.5 M H₂SO₄ per well is added to stop the reaction; plates are read at 450 nm.

In cases where the formulation of the invention comprises IFN-β any method known in the art for the detection and/or quantitation of neutralizing antibodies to IFN-β is encompassed within the methods of the invention. An exemplary method for determining the neutralizing antibodies against IFN-β may comprise the following: samples of rat serum known to contain antibody against rat IFN-β are analyzed in a cytopathic effect (CPE) inhibition assay. Cytopathic inhibition assays are known to one skilled in the art and are disclosed for example in Rubinstein et al, 1981, J. Virol. 37: 755-758; Rubinstein et al., 1981, Methods in Enzymology, (Pestka, ed.), Academic Press, New York, pp. 387-394; both of which are incorporated herein by reference in their entireties. An exemplary CPE assay comprises the following: a cell line (e.g., L929 fibroblast) which is susceptible to infection with Vesicular Stomatitis Virus (VSV), is cultured in the presence of the antibody of interest, IFN-β and VSV; comparison of cell viability is made against a culture containing only L929 cells and VSV and another culture containing L929 cells, VSV and IFN-β. In the culture containing L929 cells and VSV, the virus will infect the cells and cell death (CPE) will be observed. In the culture containing L929 cells, VSV and IFN-β, the IFN-β will block the viral binding site on the L929 cell and the cell will live (CPE inhibition). If the antibodies of interest are neutralizing against IFN-β, the IFN-β will not be able to bind the cellular epitope, the cell will be infected and cell death will be observed. In a more specific embodiment, the IFN-β neutralization assay may comprise the following: two-fold serial dilutions of the antibody-containing serum sample are prepared and 100 μL of each are added to wells of a 96 well microtiter plate; 50 μL of IFN-β are added to each well; 50 μL of L929 cell suspension is added to each well; and incubated at 37° C. for 24 hours in a 6% CO₂ controlled humidity incubator; the contents of each well are removed and the wells infected with VSV; 24 hours post-infection, the cells in the wells are fixed and stained and the plate read spectrophotometrically; the neutralizing antibody titer is the reciprocal of the highest dilution that neutralizes 50% of the IFN-β effect.

Several aspects of the formulations of the invention are preferably tested in vitro, e.g., in a cell culture system, and in an animal model organism, such as a rodent animal model system, for the desired therapeutic activity prior to use in humans. In addition to immunological based assays described supra, the invention encompasses histopathological assays to determine the effect of administering a formulation of the invention to the intradermal compartment relative to other routes of delivery such as IM and SC. An exemplary method for tissue preparation and histological evaluation using a formulation of the invention may comprise administering a formulation of the invention to the intradermal compartment of an animal subject's skin, e.g., rat; administering the same formulation either intramuscularly or subcutaneously; and analyzing tissue samples form the injection site using common pathological methods known in the art.

An exemplary method for sample preparation for pathological studies in accordance with the methods of the invention may comprise the following: upon ID administration of a formulation of the invention, the skin is removed from each injection site and placed on a small card; the excised skin is preferably approximately 1 cm in diameter with the needle insertion point in the center; the center of the injection is marked with ink at time of injection to help identify the needle insertion point; samples are placed in individual containers with 10% formalin for fixation. Upon IM injection, the needle insertion point is marked with ink at the time of injection; the skin is carefully lifted and the muscle surface directly below the needle insertion point is marked with ink; the hamstring muscle is collected and placed on a small card; samples are placed in individual containers with 10% formalin for fixation; all samples are collected preferably 24 hours post-injection. An exemplary specimen preparation may comprise the following: a single section is taken from each sample; tissue is blocked to a size appropriate for sectioning; the section is taken through the needle insertion track, Hematoxylin and Eosin (H&E) stained and evaluated. If evidence of irritation is observed, further sections should be taken to determine the extent of the irritation. Samples are graded on a multi-parameter irritancy scalesuch as that provided below. Histological alterations are graded on the following 5-grade scale:
Grade 0: Normal/Absent/None
Grade 1: Minimal/Cell Degeneration
Grade 2: Mild/Cell Necrosis
Grade 3: Moderate/Focal Erosion
Grade 4: Marked/Massive/Generalized Erosion
Present: Finding present/Severity not scored

In addition to the grading scale, descriptions of changes and findings are documented. Measurements are taken of the width and depth of any site irritation as well as skin thickness and representative photos are taken of each condition.

The methods of the invention result in minimal to moderate, preferably minimal irritation at the injection site based on histopathological evaluations such as those disclosed herein. In most preferred embodiments, the irritation at the injection site is not altered upon chronic administration. Skin reactions are assessed visually on the skin surface by scoring the appearance of the skin using the Draize scale to assess erythema and edema; other skin irritation scoring scales and methods may also be used. The methods of the invention result in no acute or chronic irritation at the site of injection as observed visually using the Draize scale.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedur may vary. Said aspects include the temporal regime of administering the prophylactic and/or therapeutic agents, and whether such agents are administered separately or as an admixture.

The anti-inflammatory activity of the therapeutic methods of invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), which is incorporated herein by reference in its entirety. Experimental and spontaneous animal models of inflammatory arthritis and autoimmune rheumatic diseases can also be used to assess the anti-inflammatory activity of the combination therapies of invention. The following are some assays provided as examples, and not by limitation.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), incorporated herein by reference in its entirety.

The anti-inflammatory activity of the therapeutic methods of invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., 2000, “Carrageenan-Induced Arthritis in the Rat,” Inflammation, 24(2): 141-155, which is incorporated herein by reference in its entirety. Also commonly used are zymosan-induced inflammation animal models as known and described in the art.

The anti-inflammatory activity of the therapeutic methods of invention can also be assessed by measuring the inhibition of carrageenan-induced paw edema in the rat, using a modification of the method described in Winter C. A. et al., 1962, “Carrageenan-Induced Edema in Hind Paw of the Rat as an Assay for Anti-inflammatory Drugs” Proc. Soc. Exp. Biol Med. 111, 544-547, which is incorporated herein by reference in its entirety. This assay has been used as a primary in vivo screen for the anti-inflammatory activity of most NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

Additionally, animal models for inflammatory bowel disease can also be used to assess the therapeutic efficacy of the methods of invention (Kim et al, 1992, Scand. J. Gastroentrol 27:529-537; Strober, 1985, Dig. Dis. Sci. 30(12 Suppl):3S-10S; both of which are incorporated herein by reference in their entireties). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for autoimmune disorders can also be used to assess the efficacy of the therapeutic methods of invention. Animal models for autoimmune disorders such as type 1 diabetes, thyroid autoimmunity, systemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24; all of which are incorporated herein by reference in their entireties).

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmacokinetic parameters of the delivered substances in accordance with the methods of the invention may be determined using methods known to one skilled in the art. An exemplary pharmacokinetic analysis of infusion data of a substance, e.g., insulin, delivered in accordance with the methods of the invention is carried out as follows. Stepwise nonlinear least-squares regression is used to analyze the insulin concentration-time data from each individual animal. Initially, an empirical biexponential equation is fit to the insulin concentration-time data for the negative control condition. This analysis assumes first-order release of residual insulin, and recovers parameters for the first-order rate constant for release, the residual insulin concentration at the release site, a lag time for release, and a first-order rate constant for elimination of insulin from the systemic circulation. The parameters recovered in this phase of the analysis are of no intrinsic importance, but merely account for the fraction of circulating insulin derived from endogenous sources.

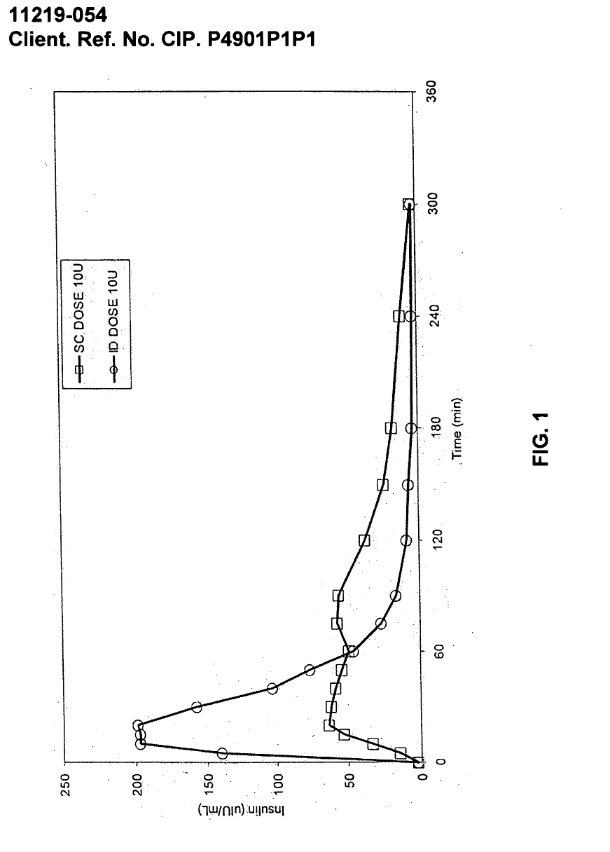

The second step of the analysis involves fitting an explicit compartmental model to the insulin concentration-time data during and after subcutaneous or intradermal infusion. The scheme upon which the mathematical model is based is shown in the upper part of FIG. 1. Infusion of insulin proceeds from t=−0 to t=240-min; after a lag-time (t_{lag,2}) absorption from the infusion site is mediated by a first-order process governed by the absorption rate constant kₐ. Insulin absorbes into the systemic circulation, distributes into an apparent volume V contaminated by an unknown fractional bioavailability F, and is eliminated according to a first-order rate constant K. The fitting routine recovered estimates of t_{lag,2}, kₐ, V/F, and K; parameters associated with the disposition of endogenous insulin (C_{R}, tl_{ag,1}, k_{R}), which are recovered in the first step of the analysis, are treated as constants. Parameter estimates are reported as mean±SD. The significance of differences in specific parameters between the two different modes of insulin administration (subcutaneous versus intradermal infusion) are assessed with the paired Student's t-test.

Pharmacodynamic analysis of insulin infusion data is calculated as follows. Plasma concentrations of glucose are used as a surrogate for the pharmacological effect of insulin. The change in response variable R (plasma glucose concentration) with respect to time t is modeled as

$$\frac{ⅆ R}{ⅆ t} = {k}_{in} - E ∘ {k}_{out}$$

where kᵢₙ is the zero-order infusion of glucose, oᵤₜ is the first-order rate constant mediating glucose elimination, and E is the effect of insulin according to the sigmoid Hill relationship

$$E = \frac{{E}_{max} ∘ {C}^{γ}}{{EC}_{50}^{γ} + {C}^{γ}}$$

in which Mₐₓ is the maximal stimulation of oᵤₜ, by insulin, EC₅₀ is the insulin concentration at which stimulation of oᵤₜ, is half maximal, C is the concentration of insulin, and y is the Hill coefficient of the relationship. Initial modeling forts utilize-the plasma concentration of-insulin as the mediator of pharmacological response. However, this approach does not capture the delay in response of plasma glucose to increasing concentrations of plasma insulin. Therefore, an effect-compartment modeling approach is finally adopted in which the effect of insulin is mediated from a hypothetical effect compartment peripheral to the systemic pharmacokinetic compartment.

The pharmacodynamic analysis is conducted in two steps. In the first step of the analysis, initial estimates of the pharmacokinetic parameters associated with the disposition of glucose (oᵤₜ, and the volume of distribution of glucose, V_{glucose}) are determined from the glucose concentration-time data in the negative control condition. The full integrated pharmacokinetic-pharmacodynamic model then is fit simultaneously to the glucose concentration-time data from the negative control condition and each insulin delivery condition for each animal (i.e., two sets of pharmacodynamic parameters are obtained for each animal: one from the simultaneous analysis of the subcutaneous insulin infusion/negative control data, and one from the simultaneous analysis of the intradermal insulin infusion/negative control data). In all pharmacodynamic analyses, the parameters governing insulin disposition obtained during pharmacokinetic analysis of insulin concentration-time data from each animal are held constant.

All other pharmacokinetic analyses are calculated using noncompartmental methods using similar software programs and techniques known in the art.

### 6. EXAMPLES

Having described the invention in general, the following specific but not limiting examples and reference to the accompanying Figures set forth various examples for practicing the dermal accessing, direct targeting drug administration method and examples of dermal administered drug substances providing improved PK and PD effects.

A representative example of dermal-access microdevice comprising a single needle were prepared from 34 gauge steel stock (MicroGroup, Inc., Medway, Mass.) and a single 28° bevel was ground using an 800 grit carborundum grinding wheel. Needles were cleaned by sequential sonication in acetone and distilled water, and flow-checked with distilled water. Microneedles were secured into small gauge catheter tubing (Maersk Medical) using UV-cured epoxy resin. Needle length was set using a mechanical indexing plate, with the hub of the catheter tubing acting as a depth-limiting control and was confirmed by optical microscopy. For experiments using :needles of various lengths, the exposed needle lengths were adjusted to 0.5, 0.8, 1, 2 or 3 mm using the indexing plate. Connection to the fluid metering device, either pump or syringe, was via an integral Luer adapter at the catheter inlet. During injection, needles were inserted perpendicular to the skin surface, and were either held in place by gentle hand pressure for bolus delivery or held upright by medical adhesive tape for longer infusions. Devices were checked for function and fluid flow both immediately prior to and post injection. This Luer Lok single needle catheter design is hereafter designated SS1_{—}34.

Yet another dermal-access array microdevices was prepared consisting of 1″ diameter disks machined from acrylic polymer, with a low volume fluid path branching to each individual needle from a central inlet. Fluid input was via allow volume catheter line connected to a Hamilton microsyringe, and delivery rate was controlled via a syringe pump. Needles were arranged in the disk with a circular pattern of 15 mm diameter. Three-needle and six-needle arrays were constructed, with 12 and 7 mm needle-to-needle spacing, respectively: All array designs used single-bevel, 34 G stainless steel microneedles of 1 mm length. The 3-needle 12 mm spacing catheter-design is hereafter designated SS3_{—}34B, 6-needle 7 mm spacing catheter-design is hereafter designated SS6_{—}34A.

Yet another dermal-access array microdevices was prepared consisting of 11 mm diameter disks machined from acrylic polymer, with a low volume fluid path branching to each individual needle from a central inlet. Fluid input was via a low volume catheter line connected to a Hamilton microsyringe, and delivery rate was controlled via a syringe pump. Needles were arranged in the disk with a circular pattern of about 5 mm diameter. Three-needle arrays of about 4 mm spacing connected to a catheter as described above. These designs are hereafter designated SS3S_{—}34_{—}1, SS3C_{—}34_{—}2, and SS3S_{—}34_{—}3 for 1 mm, 2 mm and 3 mm needle lengths respectively.

Yet another dermal-access ID infusion device was constructed using a stainless steel 30 gauge needle bent at near the tip at a 90-degree angle such that the available length for skin penetration was 1-2 mm. The needle outlet (the tip of the needle) was at a depth of 1.7-2.0 mm in the skin when the needle was inserted and the total exposed height of the needle outlet 1.0-1.2 mm This design is hereafter designated SSB1_{—}30.

### 6.1 ID Delivery of Insulin

Slow-infusion ID insulin delivery was demonstrated in swine using a hollow, silicon-based single-lumen microneedle (2 mm total length and 200×100 μm OD, corresponding to about 33 gauge) with an outlet 1.0 μm from the tip (100 μm exposed height), was fabricated using processes known in the art (U.S. Pat. No. 5,928,207) and mated to a microbore catheter (Disetronic). The distal end of the microneedle was placed into the plastic catheter and cemented in place with epoxy resin to form a depth-limiting hub. The needle outlet was positioned approximately 1 mm beyond the epoxy hub, thus limiting penetration of the needle outlet into the skin to approximately 1 mm, which corresponds to the depth of the intradermal space in swine. The catheter was attached to a MiniMed 507 insulin pump for control of fluid delivery. The distal end of the microneedle was placed into the plastic catheter and cemented in place with epoxy resin to form a death-limiting hub. The needle outlet was positioned approximately 1 mm beyond the epoxy hub, thus limiting penetration of the needle outlet into the skin to approximately 1 mm, which corresponds to the depth of the intradermal space in swine. The patency of the fluid flow path was confirmed by visual observation, and no obstructions were observed at pressures generated by a standard 1-cc syringe. The catheter was connected to an-external insulin infusion pump (MiniMed 507) via the integral Luer connection at the catheter outlet. The pump was filled with Humalog™ (Lispro) insulin (Eli Lilly, Indianapolis, Ind.) and the catheter and microneedle were primed with insulin according to the manufacturer's instructions. Sandostatin® (Sandoz, East Hanover, N.J.) solution was administered via IV infusion to anesthetized swine to suppress basal pancreatic function and insulin secretion. After a suitable induction period and baseline sampling, the primed microneedle was inserted perpendicular to the skin surface in the flank of the animal up to the hub stop. Insulin infusion at a rate of 2 U/hr was used and maintained for 4 hr. Blood samples were periodically withdrawn and analyzed for serum insulin concentration and blood glucose values. Baseline insulin levels before infusion were at the background detection level of the assay. After initiation of the infusion, serum insulin levels showed an increase that was commensurate with the programmed infusion rates. Blood glucose levels also showed a corresponding drop relative to negative controls (NC) without insulin infusion and this drop was improved relative to conventional SC infusion. In this experiment, the microneedle was demonstrated to adequately breach the skin barrier and deliver a drug in vivo at pharmaceutically relevant rates. The ID infusion of insulin was demonstrated to be a pharmacokinetically acceptable administration route, and the pharmacodynamic response of blood glucose reduction was also demonstrated. Calculated PK parameters for ID infusion indicate that insulin is absorbed much faster than via than SC administration. Absorption from the ID space begins almost immediately: the lag time prior to absorption (t_{lag}) was 0.88 vs. 13.6 min for ID and SC respectively. Also the rate of uptake from the administration site is increased by approximately 3-fold, kₐ=0.0666 vs. 0.0225 min⁻¹ for ID and SC respectively. The bioavailability of insulin delivered by ID administration is increased approximately 1.3 fold greater than SC administration.

### 6.2 Bolus Delivery of Lispro Insulin

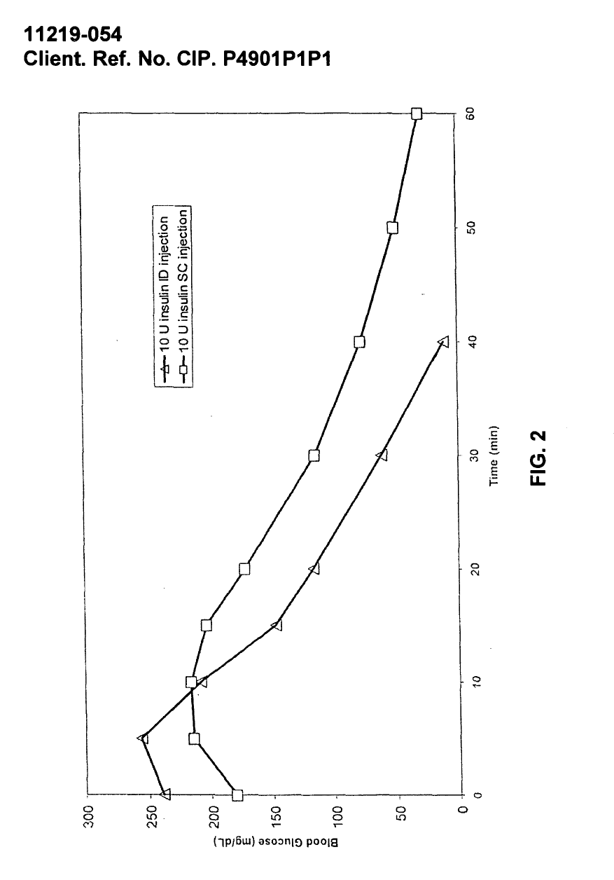

Bolus delivery of Lilly Lispro fast acting insulin was performed using ID and SC bolus administration. The ID injection microdevice was dermal access array design SS3_{—}34.10 international insulin units (U) corresponding to 100 μL volume respectively, were administered to diabetic Yucatan Minis swine. Test animals had been previously been rendered diabetic by chemical ablation of pancreatic islet cells, and were no longer able to secrete insulin. Test animals received their insulin injection either via the microneedle array or via a standard 30 G×½ in. needle inserted laterally into the SC tissue space. Circulating serum insulin levels were detected using a commercial chemiluminescent assay kit (Immulite, Los Angeles, Calif.) and blood glucose values were determined using blood glucose strips. ID injections were accomplished via hand pressure using an analytical microsyringe and were administered over approximately 60 sec. By comparison, SC dosing required only 2-3 sec. Referring to FIG. 1, it is shown that serum insulin levels after bolus administration demonstrate more rapid uptake and distribution of the injected insulin when administered via the ID route. The time to maximum concentration (Tₘₐₓ) is shorter and the maximum concentration obtained (Cₘₐₓ) is higher for ID vs. SC administration. In addition, FIG. 2 also demonstrates the pharmacodynamic biological response to the administered insulin, as measured by the decrease in blood glucose (BG), showed faster and greater changes in BG since more insulin was available early after ID administration.

### 6.3 ID Delivery of Insulin Lispro and Hoechst Regular Insulin

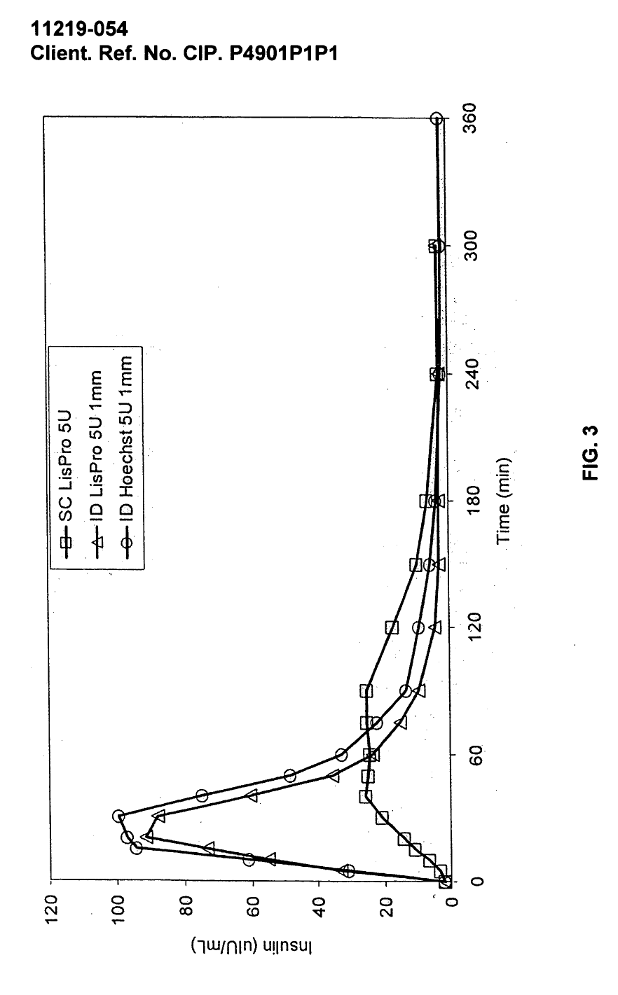

Lilly Lispro is regarded as fast acting insulin, and has a slightly altered protein structure relative to native human insulin. Hoechst regular insulin, maintains the native human insulin protein structure that is chemically similar, but has slower uptake than Lispro when administered by the traditional SC route. Both insulin types were administered in bolus via the ID route to determine if any differences in uptake would be discemable by this route. 5 U of either insulin type were administered to the ID space using dermal access microdevice design SS3_{—}34. The insulin concentration verses time data shown in FIG. 3. When administered by the ID route the PK profiles for regular and fast-acting insulin were essentially identical, and both insulin types exhibited faster uptake than Lispro given by the traditional SC route. This is evidence that the uptake mechanism for ID administration is less affected by minor biochemical changes in the administered substance, and that ID delivery provides an advantageous-PK uptake profile for regular insulin that-is superior to SC administered fast-acting insulin.

### 6.4 Bolus Delivery of Insulin Lispro Via Various Needle Length

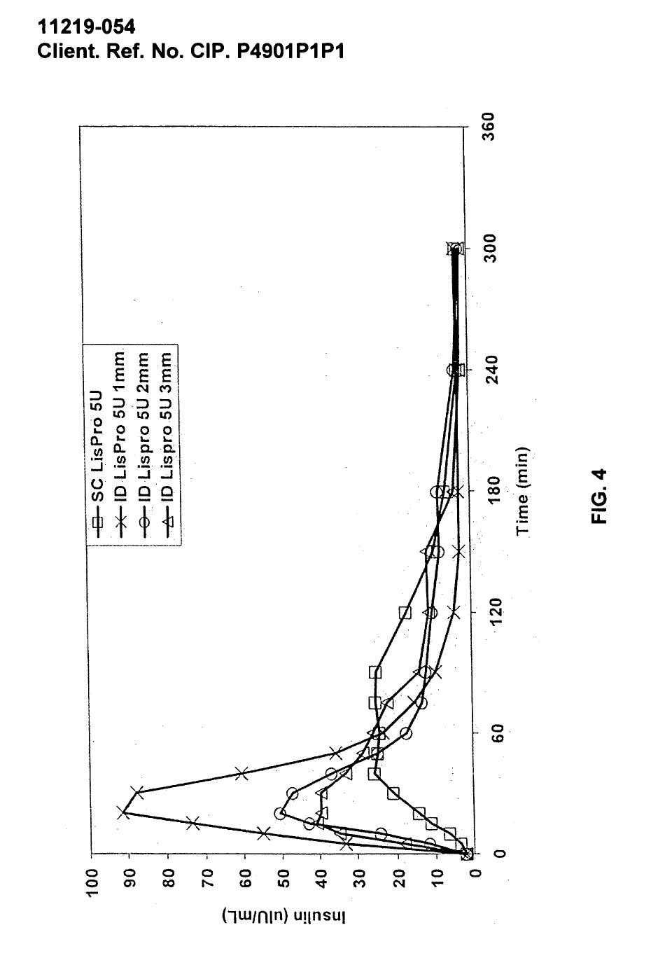

Bolus delivery of Lilly Lispro fast-acting insulin via microneedle arrays having needles of various lengths was conducted to demonstrate that the precise deposition of drug into the dermal space is necessary to obtain the PK advantages and distinctions relative to SC. Thus, 5 U of Lilly Lispro fast-acting insulin was administered using dermal access design SS3_{—}34. Additional microdevices of the same needle array configuration were fabricated whereby exposed needle lengths of the microdevice array, were lengthened to include arrays with needles lengths of 2 and 3 mm. The average total dermal thickness in Yucatan Mini swine ranges from 1.5-2.5 mm. Therefore insulin deposition is expected to be into the dermis, approximately at the dermal/SC interface, and below the dermis and within the SC for 1 mm, 2 mm, and 3 mm length needles respectively. Bolus insulin administration was as described in Example 6.2. Average insulin concentrations verses time are shown in FIG.4. The data clearly shows as microneedle length is increased, the resulting PK profile begins to more closely resemble SC administration. This data demonstrates the benefits of directly targeting the dermal space, such benefits include rapid uptake and distribution, and high initial concentrations. Since the data are averages of multiple examples, they do not show the increased inter-individual variability in PK profiles from longer 2 and 3 mm microneedles. This data demonstrates that since skin thickness may vary both between individuals and even within a single individual, shorter needle lengths that accurately target the dermal space are more reproducible in their PK profile since they are depositing the drug more consistently in the same tissue compartment. This data demonstrates longer microneedles that deposit or administer substances deeper into the dermal space, or partially or wholly into the SC space, mitigate or eliminate the PK advantages in comparison to shallow, directly targeted administrations to the highly vascularized dermal region.

### 6.5 Bolus Delivery of Lantus Long-Acting Insulin

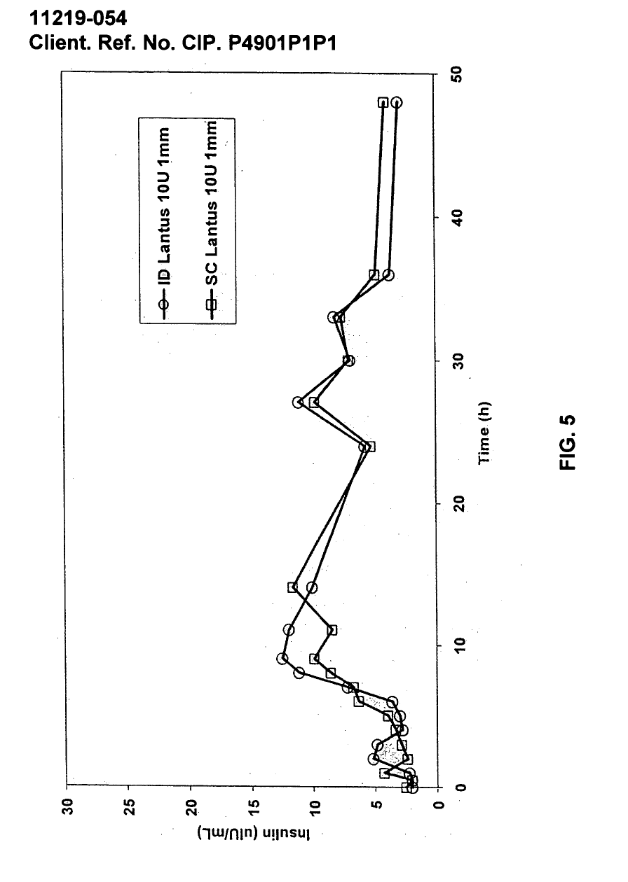

Bolus delivery of Lantus long-acting insulin was delivered via the ID route Lantus is an insulin solution that forms microprecipitates at the administration site upon injection. These microparticulates undergo slow dissolution within the body to provide (according to the manufacturer's literature) a more stable low level of circulating insulin than other current long-acting insulin such as crystalline zinc precipitates (e.g., Lente, NPH). Lantus insulin (10 U dose, 100 μL) was administered to diabetic Yucatan Mini pigs using the dermal access design SS3_{—}34 and by the standard SC method as previously described. Referring to FIG. 5, when administered via the ID route, similar PK profiles were obtained relative to SC. Minor distinctions include a slightly higher “burst” immediately after the ID insulin delivery. This demonstrates that the uptake of even very high molecular weight compounds or small particles is achievable via ID administration. More importantly this supports the fact that the biological clearance mechanism in the body is not appreciably changed by the administration route, nor is the way in which that the drug substance is utilized. This is extremely important for drugs compounds that have a long circulating half-life (examples would be large soluble receptor compounds or other antibodies for cancer treatment, or chemically modified species such as PEGylated drugs).

### 6.6 Bolus ID Delivery of Human GCSF

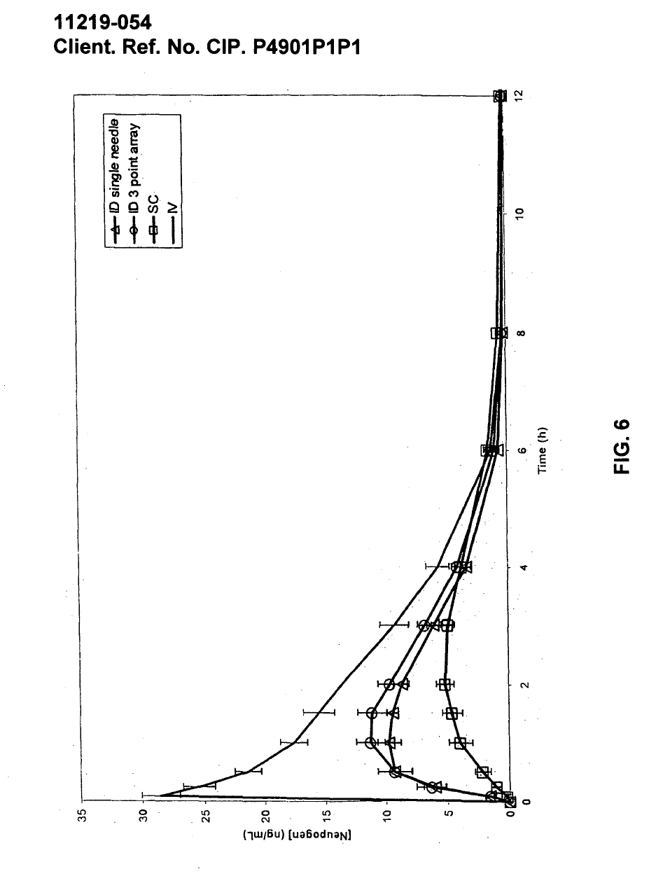

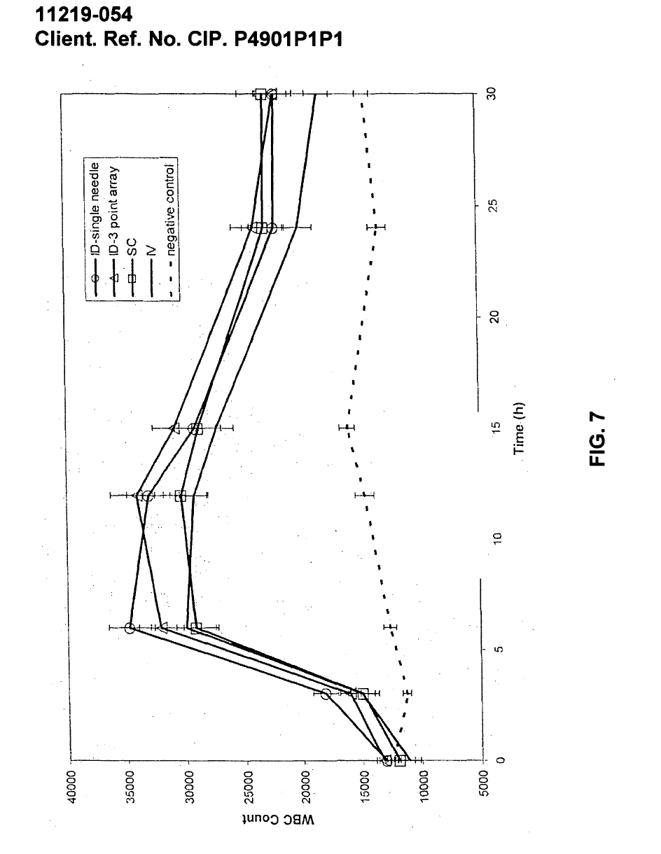

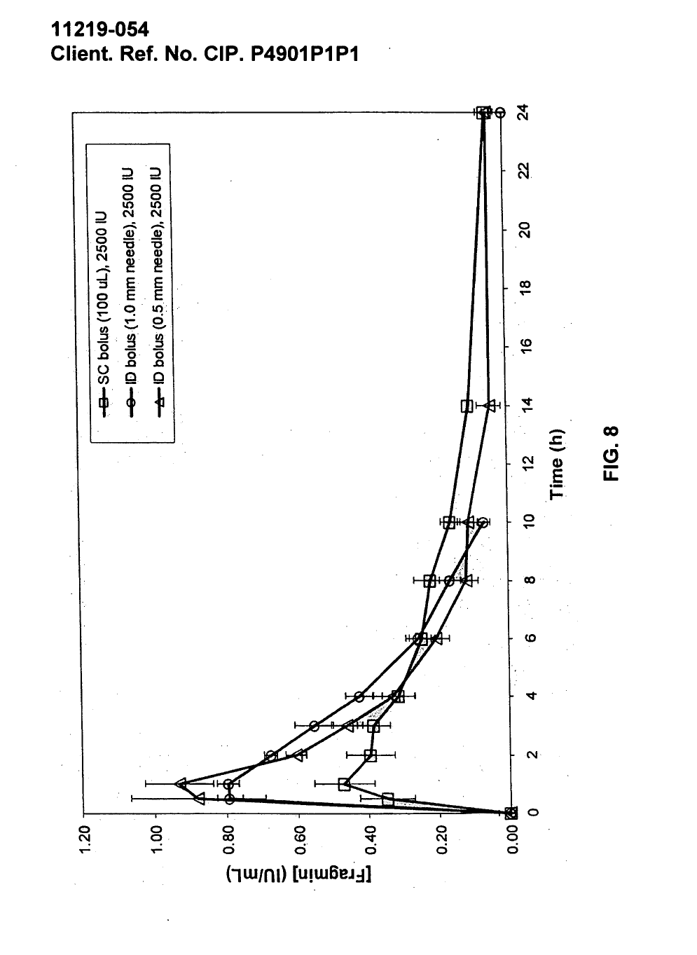

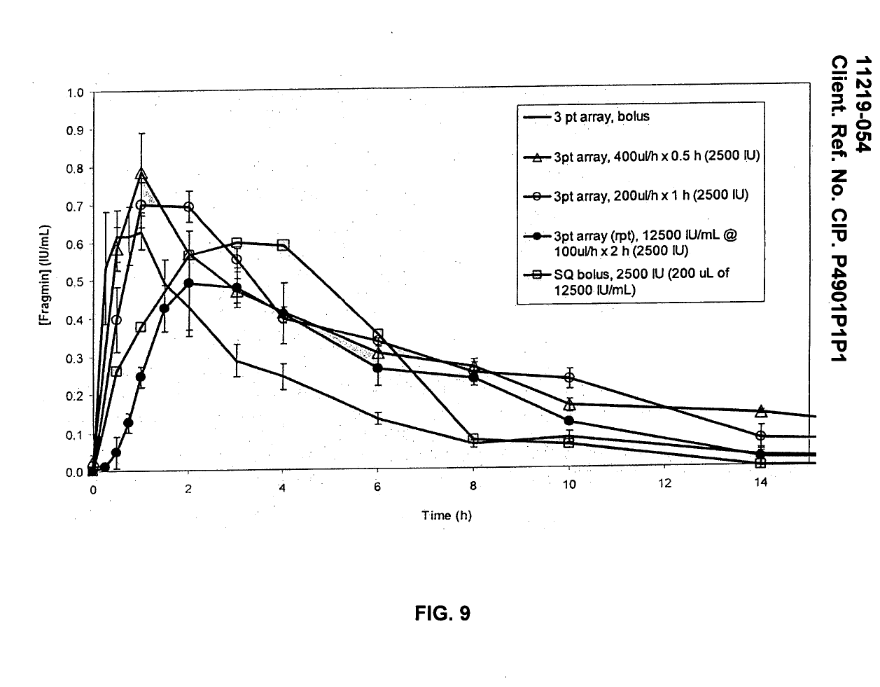

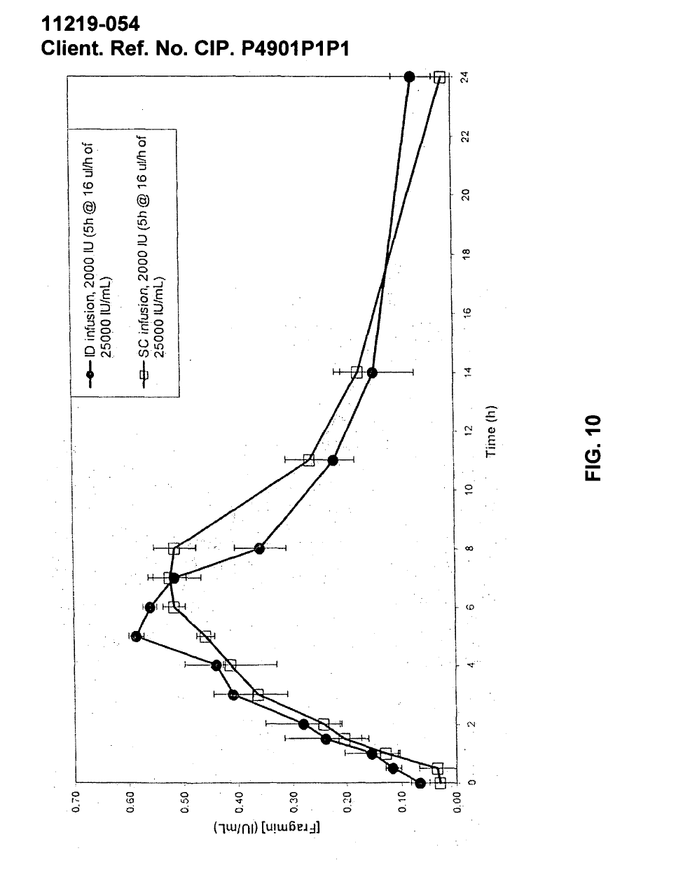

Bolus ID delivery of human granulocyte colony stimulating factor (GCSF) (Neupogen) was administered via dermal access microdevice designs SS3_{—}34B (array) or SS1_{—}34 (single needle) to Yucatan minipigs. Delivery rate was controlled via a Harvard syringe pump and was administered over a 1-2.5 min period. FIG. 6 shows the PK availability of GCSF in blood plasma as detected by an ELISA immunoassay specific for GCSF. Administration via IV and SC delivery was performed as controls. Referring to FIG. 6 bolus ID delivery of GCSF shows the more rapid uptake associated with ID delivery. Cₘₐₓ is achieved at approximately 30-90 minutes vs. 120 min for SC. Also the bioavailability is dramatically increased by an approximate factor of 2 as evidenced by the much higher area under the curve (AUC). Circulating-levels of GCSF are detectable for an extended period; indicting that ID delivery does not alter the intrinsic biological clearance mechanism or rate for the drug. These data also show that device design has minimal effect on the rapid uptake of drug from the ID space. The data referred to in FIG. 7 also shows the degree and time course of white blood cell expansion as a result of GCSF administration with respect to a negative control (no GCSF administered). White blood cell (WBC) counts were determined by standard cytometric clinical veterinary methods ID delivery exhibits the same clinically significant biological outcomes. Although all delivery means give approximately equal PD outcomes, this data suggests ID delivery could enable use half the dose to achieve essentially the same physiological result in comparison to SC, due to approximately 2-fold bioavailability increase.

### 6.7 ID Delivery of PTH

An ID administration experiment was conducted using a peptide drug entity: human parathyroid hormone 1-34 (PTH). PTH was infused for a 4 hour period, followed by a 2 hour clearance. Control SC infusion was through a standard 31-gauge needle inserted into the SC space lateral to the skin using a “pinch-up” technique. ID infusion was through dermal access microdevice design SSB1_{—}30 (a stainless steel 30-gauge needle bent at the tip at a 90° angle such that the available length for skin penetration was 1-2 mm). The needle outlet (the tip of the needle) was at a depth of 1.7-2.0 mm in the skin when the needle was inserted. A 0.64 mg/mL PTH solution was infused at a rate of 75 μL/hr. Flow rate was controlled via a Harvard syringe pump. {The weight normalized delivery profiles show a larger area under the curve (AUC) indicating higher bioavailability, higher peak values at earlier sampling timepoints (e.g., 15 and 30 min) indicating more rapid onset from ID delivery, and rapid decrease following termination of infusion (also indicative of rapid uptake without a depot effect).}

The above examples and results demonstrate the inventive delivery method using multi-point array ID administration and single needle ID administration results in more rapid uptake with higher Cₘₐₓ than SC injection. ID uptake and distribution is ostensibly unaffected by device geometry parameters, using needle lengths of about 0.5 to about 1.7 mm, needle number and needle spacing. No concentration limit for biological absorption was found and PK profiles were dictated principally by the concentration-based delivery rate. The primary limitations of ID administration are the total volume and volumetric infusion-rate limits for leak-free instillation of exogenous substances into a dense tissue compartment. Since absorption of drugs from the ID space appears to be insensitive to both device design and volumetric infusion rate, numerous formulation/device combinations can be used to overcome this limitations and provide the required or desired therapeutic profiles. For example, volume limited dosing regimens can be circumvented either by using more concentrated formulations or increasing the total number of instillation sites. In addition, effective PK control is obtained by manipulating infusion or administration rate of substances.

In general, ID delivery as taught by the methods described hereto via dermal access microneedle devices provides a readily accessible and reproducible parenteral delivery route, with high bioavailability, as well as the ability to modulate plasma profiles by adjusting the device infusion parameters, since uptake is not rate-limited by biological uptake parameters.

In the previously described examples, the methods practiced by the invention demonstrate the ability to deliver a drug in vivo with greatly improved pharmaceutically relevant rates. This data indicates an improved pharmacological result for ID administration as taught by the methods described of other drugs in humans would be expected according to the methods of the invention.

### 6.8 ID Admistration of IFN-β Versus IM Administration

### 6.8.1 Antibody Titer Studies

MATERIALS AND METHODS: Antibody titers were evaluated using an ELISA assay. 100 μL of 1 μg/mL of rat interferon beta (Cell Sciences Inc. (Canton, Mass.)) in coating buffer was added to the wells of a 96 well plate. The plate was incubated at 4° C. overnight. Well contents were removed and the wells were blocked with 5% skim milk in PBST for 2 hours at 37° C. Wells were washed and 100 μL of serum samples were added to the first row of wells. Samples were serially diluted across the plate. The plate was incubated for 1 hour at 37° C. and washed. 100 μL of 1:4000 diluted, anti-rat HRP-conjugated antibody was added to each well and the plate was incubated for 30-60 minutes at 37° C. 100 μL of TMB color developer was added to each well. 200 μL of hydrochloric acid reaction stopper was added to each well. The plate was read at 450 nm. The antibody titer is the dilution giving 3 times absorbance of blank serum.

Isotype specific antibody responses against IFN-β were determined at weeks 2 and 24 post-injection using an ELISA Quantification Kit, commercially available from Bethyl Laboratories (Bethyl Inc., Montgomery, Tex.). A 96 well microtiter plate (Nunc Maxi-sorb plate) was coated with 1 μg/mL of IFN antigen and incubated overnight at 4° C. (to generate a standard curve wells were coated with 1 μL of capture antibody to 100 μL coating buffer); plates were washed with 50 mM Tris buffered saline, pH 8.0, 0.05% Tween (Sigma #T9039); 200 μL of Post Coat/Block solution (50 mM Tris buffered saline, pH 8.0, 1% BSA; Sigma #T6789) was added per well and incubated covered for 30 minutes at room temperature. Standards were prepared according to the package insert using the Sample/Conjugate Diluent (0.5 mL of 10% Tween 20; Sigma # P7949) per 100 mL of PostCoat/Block Solution; sample dilutions were made using the Sample/Conjugate Diluent; 100 μL of standard or sample was transferred to assigned wells and incubated for 1 hour at room temperature; conjugate was prepared according to package insert (1:20,000 for IgG1 and 1:40,000 for IgG2b); 100 μL of the conjugate was added per well and incubated for 1 hour at room temperature; 100 μL of TMB per well was added and incubated at room temperature for 30 minutes; 200 μL of 0.5 M H₂SO₄ per well was added to stop the reaction; plates were read at 450 nm.

Neutralizing antibody titers were performed by the following Interferon beta neutralization assay. Mouse 3T3 cells were seeded into wells of 96 well plates at approximately 15,000 cells/well. At least 1 day after plating, the cells were treated with IFN-β for 24 hours according to the following dilution series. Sera samples were diluted in media at 1:10, 1:100, 1:1000 and 1:10,000 in another 96 well plate in a total volume of 50 μL. 100 μL of 25 U/mL rat IFN-β was added to the wells and the plates (not including cells) were incubated for 1 to 3 hours at 4° C. The media from the wells containing cells was aspirated and 125 μL of the diluted sera sample was added to the well containing 3T3 cells. A set of controls with no sera was included. After 24 hours, 1000 plaque forming units of VSV (vesicular stomatitis virus) in 50 μL of media was added to each well. A second control as virus without IFN-β was added. After an additional 24 hours of incubation, the wells were assayed for virus induced cell death (cytopathicity of VSV) by visual inspection under a microscope. IFN-β will protect cells from VSV-induced cell death, and if there is neutralizing activity in the sera it will neutralize IFN-β's antiviral effect. All assays were performed twice in duplicate.

STUDY DESIGN: A chronic dosing regimen was performed in female Wistar rats using recombinant rat IFN-β (Cell Sciences Inc. (Canton, Mass.)). An injection route comparison was performed evaluating the immunogenicity of IFN-β upon injection intradermally or intramuscularly. IFN-β was injected intradermally using BD's 34 Ga, 1 mm needle and injected intramuscularly using BD 30 Ga×0.5″. Animals were dosed in the quadriceps for the first 6 weeks of the study; and then into the hamstring for remaining months of the study. IFN-β was administered at the doses indicated in Table 2 below, which are the typical doses used in humans, 3 times a week for 6 months. Blood samples were collected once a week.

TABLE 2
THERAPY REGIMEN

| WEEK | DOSE | DOSAGE |
|---|---|---|
| 1-2 | 8.8 μg | 0.129 μg/kg |
| 3-4 | 22 μg | 0.324 μg/kg |
| 5-on | 44 μg | 0.647 μg/kg |

The study design is shown in Table 3 below:

TABLE 3
STUDY DESIGN

| N | 10 |
|---|---|
| Group I | IM injection |
| Group II | ID injection |
| Dose | 3 times/wk for 24 weeks |
| Regimen | |
| Sampling | Blood samples taken retroorbitally |
| Regimen | once a week |
| Dose Vol. | 50 μL |
| Dosing | Weeks 1-2: 0.129 μg/kg |
| Regimen | Weeks 3-4: 0.324 |
| | Weeks 5 and on: 0.647 |
| | Serum Analysis: ELISA |

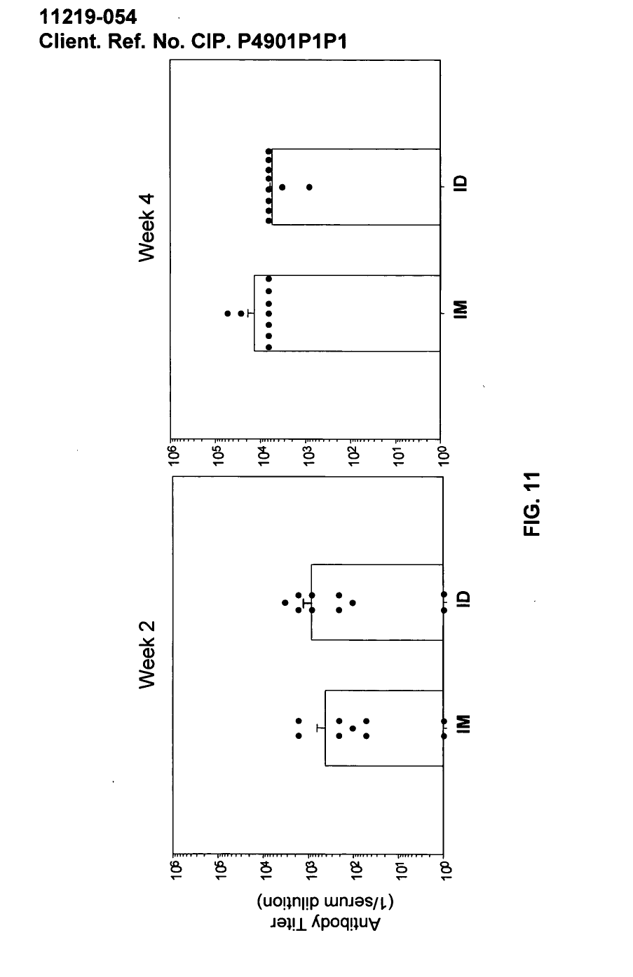

RESULTS: Antibody titers (as determined by ELISA) of the 2^{nd} week bleed, 4^{th} week bleed, 6^{th} week bleed, 12^{th} week bleed, and 24^{th} week bleed are shown in FIGS. 11-13, respectively. Antibody titer (as determined by ELISA) of the 18^{th} week bleed is shown in Table 4 below.

TABLE 4
Antibody Titers: Week 18. Titer is defined as the highest dilution of a
serum sample yielding an absornance value that is at least 3X background
obtained from naïve, undosed animals.

| IM TITERS | ID TITERS |
|---|---|
| 819200 | 102400 |
| 819200 | 51200 |
| 409600 | <25600 |
| <25600 | <25600 |
| <25600 | <25600 |
| <25600 | <25600 |
| <25600 | <25600 |
| <25600 | <25600 |
| Died | <25600 |
| Died | <25600 |

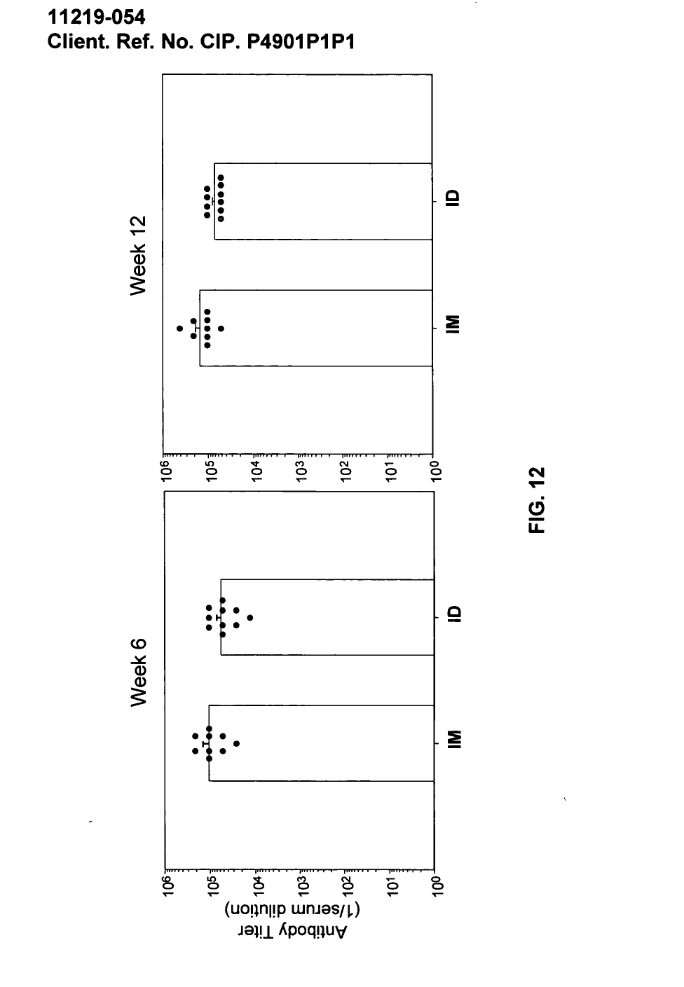

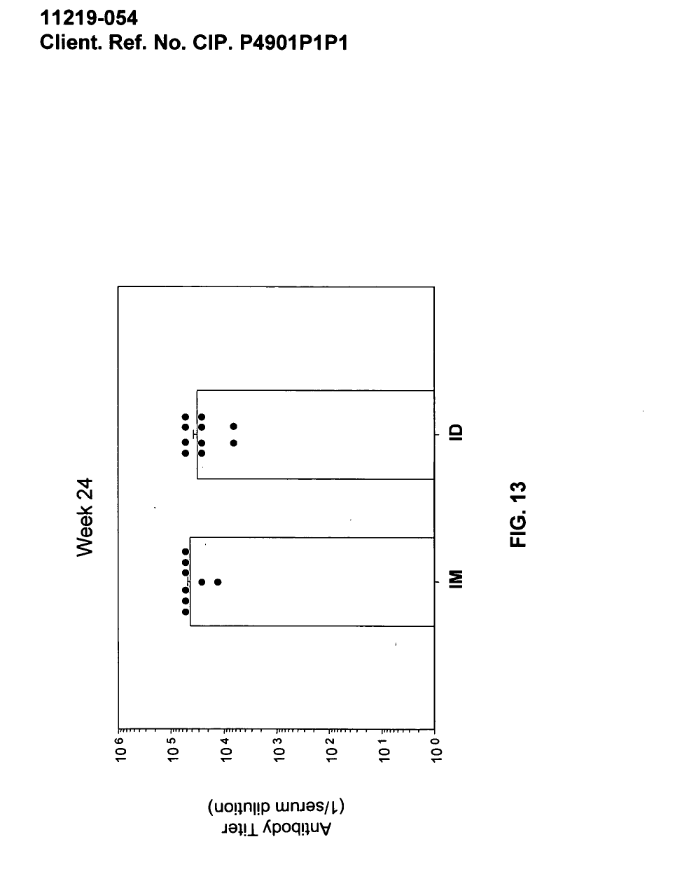

As seen in FIGS. 11-13, IM and ID injection-induced antibody titers by ELISA were not significantly different at weeks 2 and 4. ID injection induced a slightly stronger antibody response initially after two weeks, however, by the 4^{th} week, the IM injection showed a higher mean antibody response (See FIGS. 12 and 13). At 6 weeks, IM injection continued to exhibit slightly higher antibody response than ID. Results from a two sample t-test indicated that after 12 weeks of dosing, the mean for IM injection was significantly greater than the mean for ID injection at the 0.05% level. As shown in Table 4, there was a general reduction in antibody response at week 18. Three animals that were injected IM showed a marked increase in titer from week 12; only one animal that had been injected ID showed an increase in titer from week 12. As shown in FIG. 13, results from a two sample t-test showed that after 24 weeks of dosing, the mean antibody titers by ELISA were not significantly different.

The data confirm that chronic dosing with same species IFN-β via ID injection is less immunogenic than dosing of the same species IFN-β via IM injection. Since it has been previously demonstrated that chronic SC injection of IFN-β is more immunogenic than IM, ID injection likely does not induce a stronger immune response than via SC injection and may induce a weaker response than via SC injection

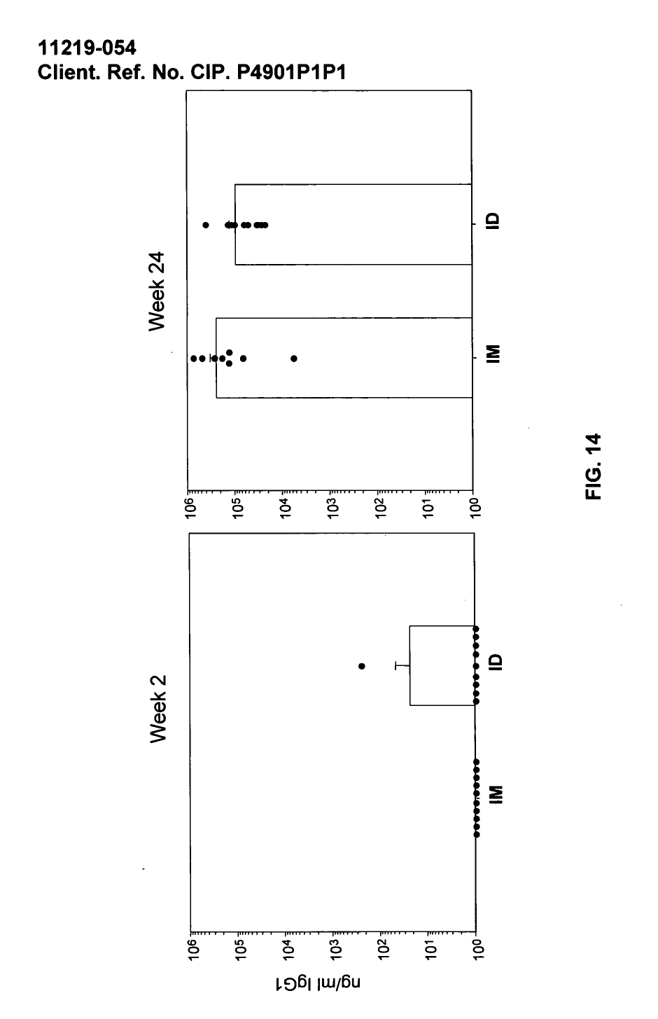

FIG. 14 shows the IgG1 antibody isotype level at weeks 2 and 24 post-injection. After the second week, only one animal showed any detectable IgG1 antibody from an ID administration. After week 24, mean IFN-β specific IgG1 concentrations were not significantly different between IM and ID injections (p<0.05).

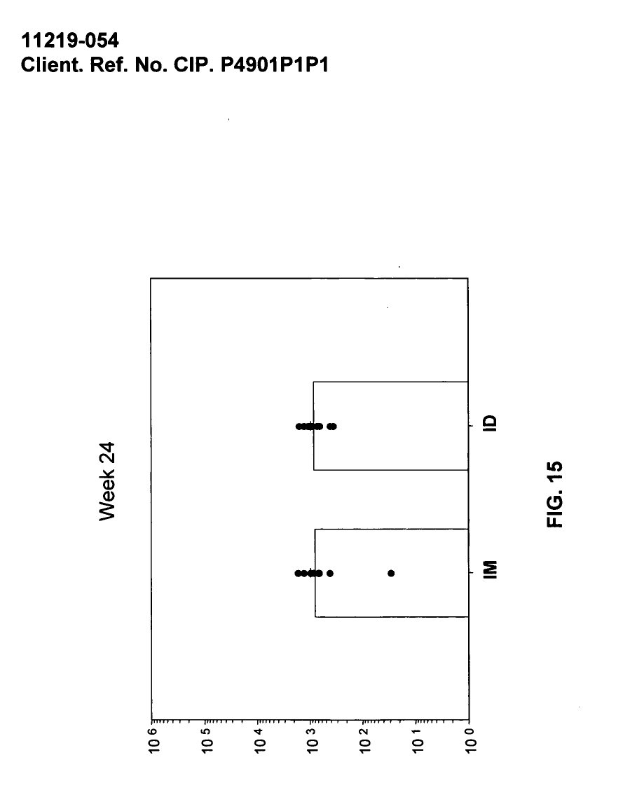

FIG. 15 shows the IgG2b antibody isotype level at week 24 post-injection. After the second week, no IgG2b was detectable regardless of the route of administration (IM or ID). After week 24, mean IFN-β specific IgG2b concentrations were not significantly different between IM and ID injections (p<0.05). The IFN-β specific IgGM concentrations were not analyzed due to high initial IgG titers by ELISA.

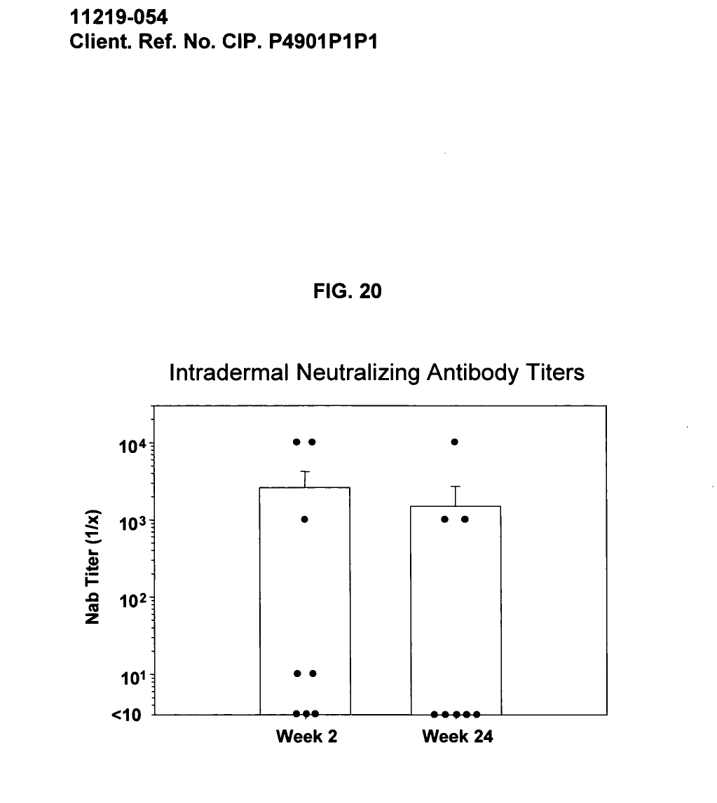

FIG. 20 shows the results of the neutralizing antibody titer assay by ELISA. At 2 weeks, 5 of the 8 animals demonstrated detectable levels of IFN-β specific neutralizing antibodies (NAbs). At 24 weeks, 3 of the 8 animals demonstrated detectable levels of IFN-β specific neutralizing antibodies (NAbs). None of the animals in the intramuscular groups demonstrated any detectable levels of NAbs. Mean IFN-β specific NAs titers by ELISA were not significantly different between IM and ID injection at either 2 or 24 weeks. Results from a two-tailed, paired t-test shows that at week 2, the t-value was 0.147; and at week 24, the t-value was 0.0796. The critical t-value (p<0.05; df=7) was 2.36. Means were not significantly different.

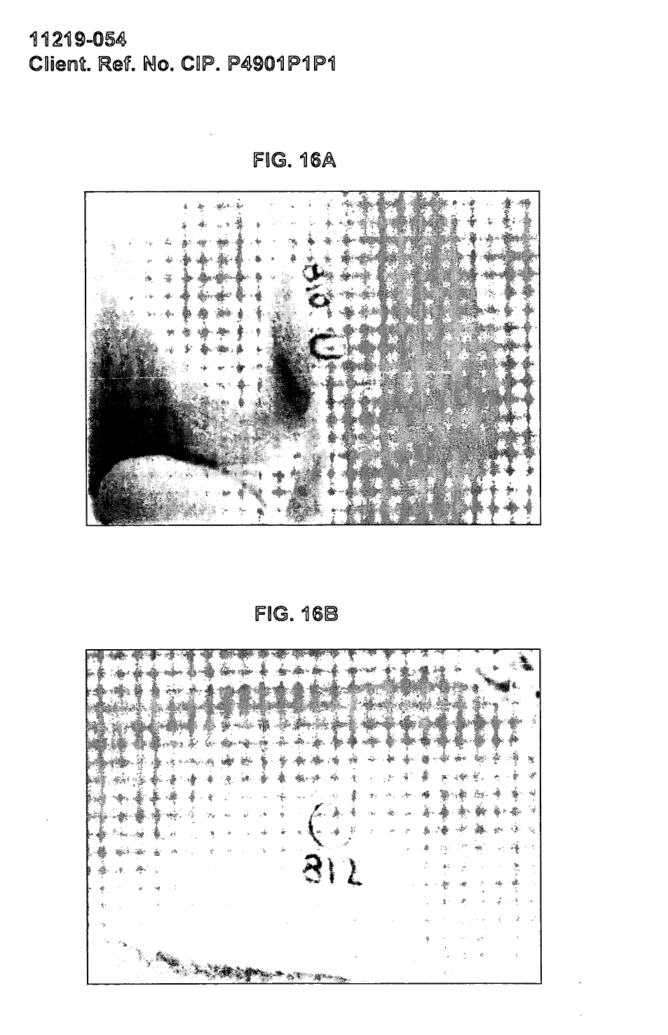

In summary, the antibody responses from both the IM and ID injections are comparable at weeks 2, 4, 6, and 24. Antibody response from ID injection is significantly less than IM injection at week 12. The IgG1 antibody response is higher at week 2 for ID injection than for IM injection; however, the IgG1 concentration at week 24 is significantly less for ID injection than for IM injection. IgG2b responses are comparable for both IM and ID injection at week 24. No acute or chronic injection site irritation was observed upon ID administration (FIG. 16). FIGS. 16A-B show injection site photos of IFN-β to female Wistar rats. Panel A is an intramuscular injection. The back of a rat's leg is shown. The injection was made into the hamstring muscle. Panel B is an intradermal injection on the rat's back. The “bleb” is circled. No irritation is evident in either photo. These photos were taken immediately post-injection. No irritation was evident at other time points; time points were taken at 2, 4, 8, 24, 48 and 96 hours, none of which showed any external irritation.

IFN-β specific antibody responses, as determined by ELISA, from both IM and ID injection are comparable throughout the study. Animals treated ID showed IFN-β neutralizing antibody titers at both weeks 2 and 24 weeks. Intramuscular animals did not show neutralizing antibodies. IFN-β specific IgG1 and IgG2b, as determined by ELISA are not significantly different between ID and I M groups (at both 2 and 24 weeks). At week 2, there were 5/18 (27%) animals with detectable NAbs in the ID group vs. 0/18 (0%) in the IM group. AT week 24, there were 3/18 (38%) animals with detectable Nabs in the ID group vs. 0/8 (0%) in the IM group. No external acute or chronic injection site irritation have been observed. Histopathological classification of irritation for ID is minimal.

### 6.8.2 Histological Evalution of Skin Following IM and ID Administration of Interferon-Beta

Material and Methods

Sample Preparation: For ID injection, the skin was removed from each injection site and placed on a small card. The excised skin was approximately 1 cm in diameter with the needle insertion point in the center. The center of the injection was marked with black tattoo ink at time of injection to help identify the needle insertion point. Samples were placed in individual containers with 10% formalin for fixation. For IM injection, the needle insertion point was marked with black tattoo ink at the time of injection. The skin was carefully lifted and the muscle surface directly below the needle insertion point was marked with black tattoo ink. The hamstring muscle was collected and placed on a small card. Samples were placed in individual containers with 10% formalin for fixation. All samples were collected 24 hours post-injection.

Sample Identification: Samples are identified by the following system. In animals 801, 803, 804, 806, 807, 808, 809 and 810 intramuscular injections were made to the hamstring muscle of female Wistar rats. In animals 811, 812, 813, 814, 815, 816, 817, 818, 819 and 820 intradermal injections (approximately 1 mm deep) were made to the lateral dorsum of female Wistar rats.

Specimen Preparation: A single section was taken from each sample. Tissue was blocked to a size appropriate for sectioning. The section was taken through the needle insertion track. Sections were H&E stained and evaluated. If evidence of irritation was observed, further sections were taken to determine the extent of the irritation. Samples were graded on a multi-parameter irritancy scale provided below. Histological alterations were graded on the following 5-grade scale:
Grade 0: Normal/Absent/None
Grade 1: Minimal/Cell Degeneration
Grade 2: Mild/Cell Necrosis
Grade 3: Moderate/Focal Erosion
Grade 4: Marked/Massive/Generalized Erosion
Present: Finding present/Severity not scored

In addition to the grading scale, descriptions of changes and findings were documented. Measurements were taken of the Width and Depth of any site irritation as well as skin thickness. Representative photos were taken of each condition.

Study Design: Rat interferon beta (Cell Sciences Inc. (Canton, Mass.) was administered IM or ID in phosphate buffered saline using methods described supra. The animals were divided according to the following scheme: 8 rats were dosed Im and 10 were dosed ID

Rats were anesthetized with isoflurane after a mixture of Acepromazine, Xylazine and Ketamine was administered IP to sedate. Rats were allowed to wake up naturally after dosing.

Animal Preparation:

Intramuscular Group—Day 1: The hair on the back and on the back of the rear leg was clipped. Injection was performed using a BD 30 G×0.5″ needle to the hamstring muscle. 50 μL was injected. The center of the injection was marked with black tattoo ink to help aid in injection track identification. Rats were returned to their cages for recovery.

Intramuscular Group—Day 2 (24 hrs. post-injection): Rats were euthanized by CO₂ asphyxiation and the skin from rear leg was removed. The muscle directly below the needle insertion point on the skin was marked with black tattoo ink. The hamstring muscle was excised, placed on small card and stored in 10% formalin solution.

Intradermal Group—Day 1 The hair on the lateral dorsum was closely clipped. and injection was performed using a 1 mm BD microneedle. 50 μL was injected The center of the injection was marked with black tattoo ink to help aid needle track identification. Rats were returned to their cages for recovery.

Intradermal Group—Day 2 (24 hrs. post-injection) Rats were euthanized by CO₂ asphyxiation. A 1 cm diameter margin of skin (full-thickness) around the injection site was excised. Skin was placed on a small card and stored in 10% formalin solution.

Dosing: The following dosing regimen was followed for Recombinant rat Interferon Beta (Cell Sciences Inc. (Canton, Mass.)):

TABLE 5
Dosing

| | IM | ID |
|---|---|---|
| TARGET DOSE | 0.149 μg | 0.149 μg |
| TARGET | 50 μL | 50 μL |
| VOLUME | | |
| INJECTION SITE | hamstring muscle of | Lateral Dorsum |
| | rear leg | |
| DOSING | 30ga × 0.5″ BD | A 1 mm microneedle |
| PROCEDURE | needle was used to | on a 3.5” catheter |
| | perform the injection | was used. Needle |
| | | was inserted |
| | | perpendicular to skin |
| | | surface. Injection |
| | | performed manually |
| | | in 10 seconds |

Sampling

Intramuscular: The hamstring muscle was removed without cutaneous tissue and placed on a card in 10% formalin solution

Intradermal: Full thickness skin was removed, approximately 1 cm diameter around needle insertion point and placed on a card into 10% formalin solution.

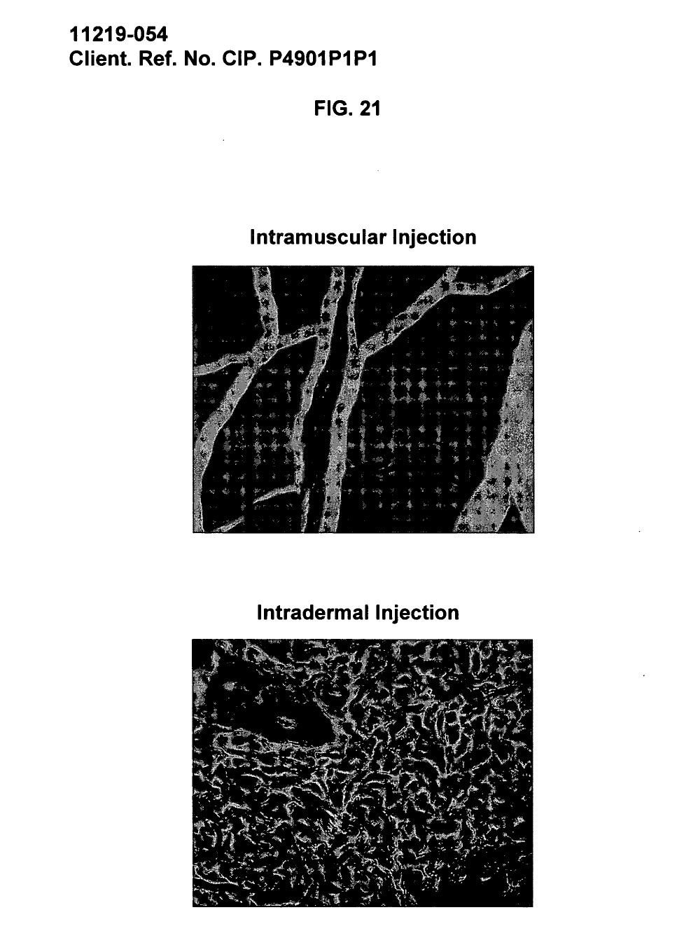

RESULTS: At 2 weeks, as shown in FIG. 21, intradermal injection sites were minimally infiltrated with lymphocytes and neutrophils. The majority of intramuscular injection sites showed inflammatory cell infiltrates that were similar to but more prominent than the intradermal sites. Intradermal injection sites were infiltrated by fewer lymphocytes and neutrophils than the intramuscular sites. The cellular reaction, in general,was minimal.

At 24 weeks, intradermal injection sites were more consistently infiltrated with lymphocytes, macrophages and neutrophils than intramuscular sites. The cellular reaction (inflammation) was minimal to moderate for ID injection and more pronounced than for IM injection. While the ID sites were more prominently affected than the IM sites, the overall irritation index would be designated as minimal. Results of the studies are summarized in the tables below

The results are summarized in the tables below.

TABLE 6
Animal No.: 811 Microscopic Classification System for Evaluation
of Intradermal Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of vessels (hemorrhage) | 4 |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 7
Animal No.: 812 Microscopic Classification System for Evaluation
of Intradermal Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of vessels (hemorrhage) | 4 |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 8
Animal No.: 813 Microscopic Classification System for Evaluation
of Intradermal Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of vessels (hemorrhage) | 4 |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 9
Animal No.: 814 Microscopic Classification System for Evaluation
of Intradermal Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of vessels (hemorrhage) | 4 |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 10
Animal No.: 815 Microscopic Classification System for Evaluation
of Intradermal Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of vessels (hemorrhage) | 4 |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 11
Animal No.: 816
Microscopic Classification System for Evaluation of Intradermal
Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of | |
| vessels (hemorrhage) | 4 |
| Edema | |
| Absent | 0 |
| Minimal | 1 |
| Mild | 2 |
| Moderate | 3 |
| Marked | 4 |
| Irritation Index | 2 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 12
Animal No.: 817
Microscopic Classification System for Evaluation of Intradermal
Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of | 4 |
| vessels (hemorrhage) | |
| Edema | |
| Absent | 0 |
| Minimal | 1 |
| Mild | 2 |
| Moderate | 3 |
| Marked | 4 |
| Irritation Index | 2 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 13
Animal No.: 818
Microscopic Classification System for Evaluation of Intradermal
Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption | 4 |
| of vessels (hemorrhage) | |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 14
Animal No.: 819
Microscopic Classification System for Evaluation of Intradermal
Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption of | 4 |
| vessels (hemorrhage) | |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

TABLE 15
Animal No.: 820
Microscopic Classification System for Evaluation of Intradermal
Delivery Devices

| Reaction | Numerical Grading |
|---|---|
| Epidermis | |
| Normal, intact | 0 |
| Cell degeneration | 1 |
| Cell necrosis | 2 |
| Focal erosion | 3 |
| Generalized erosion | 4 |
| Dermis | |
| Leukocyte infiltration (per 40x field) | |
| Absent | 0 |
| Minimal - <25 | 1 |
| Mild - 25-50 | 2 |
| Moderate - 51-100 | 3 |
| Marked - >100 | 4 |
| Vascular Congestion | |
| Absent | 0 |
| Minimal | 1 |
| Mild | 2 |
| Moderate | 3 |
| Marked, with disruption | 4 |
| of vessels (hemorrhage) | |
| Edema | |
| Absent | 0 |
| Minimal | 1 |
| Mild | 2 |
| Moderate | 3 |
| Marked | 4 |
| Irritation Index | 3 |

| Average Score | Adjectival Description |
|---|---|
| 0 | None |
| 1 to 4 | Minimal |
| 5 to 8 | Mild |
| 9 to 11 | Moderate |
| 12 to 16 | Severe |

All of the patents, patent applications and references referred to in this application are incorporated in their entirety by reference. Moreover, citation or identification of any reference in this application is not an admission that such reference is available as prior art to this invention. The full scope of this invention is better understood with reference to the appended claims.

1. A method for administration of a substance to a subject's skin comprising delivering the substance into an intradermal compartment of the subject's skin, wherein the substance results in an immune response no greater than when the substance is delivered intramuscularly.

2. A method for administration of a substance to a subject's skin comprising delivering the substance into an intradermal compartment of the subject's skin, wherein the substance results in an immune response no greater than when the substance is delivered subcutaneously.

3. A method for administration of a substance to a subject's skin comprising delivering the substance into an intradermal compartment of the subject's skin, wherein the substance elicits an immune response that is of the same type as that generated when the substance is administered to the intramuscular or subcutaneous compartment of the subject's skin.

4. A method for administration of a substance to a subject's skin comprising delivering the substance into an intradermal compartment of the subject's skin, wherein the substance elicits an immune response that is of a different type as that generated when the substance is administered to the intramuscular or subcutaneous compartment of the subject's skin.

5. The method of any of claims 1-4, wherein the substance is a therapeutic or diagnostic substance.

6. The method of any of claims 1-4, wherein the therapeutic substance is a chemokine, cytokine or an immunomodulatory agent.

7. The method of any of claims 1-4, wherein the cytokine is interferon.

8. The method of any of claims 1-4, wherein the interferon is interferon-α, interferon-β or interferon-γ.

9. A method for treating or preventing multiple sclerosis in a subject comprising delivering a therapeutically or prophylactically effective amount of an IFN-β formulation to an intradermal compartment of the subject's skin.

10. The method of any of claims 1-4, wherein the substance is administered at least once a week over a time period of at least 4 weeks, at least 6 weeks, at least 2 months, or up to the life time of the subject.

11. The method of any of claims 1-4, wherein the substance is administered at least twice a week over a time period of at least 4 weeks, at least 6 weeks, at least 2 months, or up to the life time of the subject.

12. The method of any of claims 1-4, wherein the substance is administered through at least one small gauge needle having an outlet with an exposed height between 0 and 1 mm, said outlet being inserted into the skin at a depth of between 0.3 mm and 2 mm, such that administration occurs at a depth between 0.3 mm and 2 mm.
